(19) United States
(12) Patent Application Publication
(10) Pub. No.: US 2004/0192753 A1
(43) Pub. Date: Sep. 30, 2004

(54) Methods of use of thrombin receptor antagonists

(76) Inventors: Samuel Chackalamannil, Califon, NJ (US); Yan Xia, Edison, NJ (US); Enrico P. Veltri, Princeton, NJ (US); Mariappan V. Chelliah, Edison, NJ (US); Wenxue Wu, Princeton Junction, NJ (US); Michael P. Graziano, Scotch Plains, NJ (US); Teddy Kosoglou, Jamison, PA (US); Madhu Chintala, Colts Neck, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 10/705,282

(22) Filed: Nov. 10, 2003

Related U.S. Application Data
(63) Continuation-in-part of application No. 09/880,222, filed on Jun. 13, 2001, now Pat. No. 6,645,987.
(63) Continuation-in-part of application No. 10/412,982, filed on Apr. 14, 2003.
(60) Provisional application No. 60/211,724, filed on Jun. 15, 2000.
(60) Provisional application No. 60/373,072, filed on Apr. 16, 2002.

(51) Int. Cl. A61K 31/404; A61K 31/365

(52) U.S. Cl. 514/411; 514/418; 514/470

(57) ABSTRACT

A method of treating a therapeutic condition comprising administering to a mammal in need of such treatment an effective amount of at least one compound of the formulas:

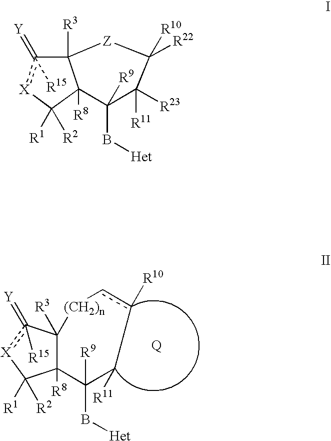

or a pharmaceutically acceptable isomer, salt, solvate or co-crystal form thereof, wherein the substituents are as defined in the specification,

wherein said therapeutic condition is a cardiovascular or circulatory disease or condition, an inflammatory disease or condition, a respiratory tract or disease or condition, cancer, acute renal failure, astrogliosis, a fibrotic disorder of the liver, kidney, lung or intestinal tract, Alzheimer's disease, diabetes, diabetic neuropathy, rheumatoid arthritis, neurodegenerative disease, neurotoxic disease, systemic lupus erythematosus, multiple sclerosis, osteoporosis, glaucoma, macular degeneration, psoriasis, radiation fibrosis, endothelial dysfunction, a wound or a spinal cord injury, or a symptom or result thereof. Combination therapy with other therapeutically effective agents is also disclosed.

### CROSS REFERENCE TO RELATED APPLICATIONS

The present application is a Continuation in Part of: 1) a co-pending U.S. Continuation in Part Application filed Sep. 25, 2003, claiming the benefit under 35 U.S.C. §119(e) of U.S. application Ser. No. 09/880222, filed Jun. 13, 2001, which claims the benefit of U.S. Provisional Application No. 60/211,724, filed Jun. 15, 2000; and 2) U.S. application Ser. No. 10/412,982, filed Apr. 14, 2003, which claims the benefit of U.S. Provisional Application No. 60/373,072, filed Apr. 16, 2002, the complete text and claims of which are incorporated by reference herein as if fully set forth.

### BACKGROUND OF THE INVENTION

Thrombin is known to have a variety of activities in different cell types and thrombin receptors are known to be present in such cell types as human platelets, vascular smooth muscle cells, endothelial cells and fibroblasts. It is therefore possible that thrombin receptor antagonists, also known as protease activated receptor (PAR) antagonists will be useful in the treatment of thrombotic, inflammatory, atherosclerotic and fibroproliferative disorders, as well as other disorders in which thrombin and its receptor play a pathological role.

Thrombin receptor antagonists peptides have been identified based on structure-activity studies involving substitutions of amino acids on thrombin receptors. In Bernatowicz et al, J. Med. Chem., vol. 39, pp. 4879-4887 (1996), tetra- and pentapeptides are disclosed as being potent thrombin receptor antagonists, for example N-trans-cinnamoyl-p-fluoroPhe-p-guanidinoPhe-Leu-Arg-NH₂ and N-trans-cinnamoyl-p-fluoroPhe-p-guanidinoPhe-Leu-Arg-Arg-NH₂. Peptide thrombin receptor antagonists are also disclosed in WO 94/03479, published Feb. 17, 1994.

Thrombin receptor antagonist have been suggested in the literature as being potentially useful in treating a variety of diseases or conditions including, for example, thrombosis, vascular restenosis, deep venous thrombosis, lung embolism, cerebral infarction, heart disease, disseminated intravascular coagulation syndrome, hypertension, inflammation, rheumatism, asthma, glomerulonephritis, osteoporosis, neuropathy and/or malignant tumors (Suzuki, Shuichi, PCT Int. Appls. WO 0288092 (2002), WO 0285850 (2002) and WO 0285855 (2002)), arrhythmia, inflammation, angina, stroke, atherosclerosis, ischemic conditions, angiogenesis related disorders, cancer, and neurodegenerative disorders (Zhang, Han-cheng, PCT Int. Appl. WO 0100659 (2001), WO 0100657(2001) and WO 0100656 (2001)), disorders of the liver, kidney and lung (Chambers, R. C., “Coagulation cascade proteases and tissue fibrosis,” Biochemical Society Transactions, 2002, 30(2), pp. 194-200), cancer (Nguyen, Quang-De, “RhoA- and RhoD-dependent regulatory switch of Gα subunit signaling by PAR-1 receptors in cellular invasion,” FASEB Journal, 2002, 16(6), pp. 565-576), melanoma (Tellez, Carmen, “Role and regulation of the thrombin receptor (PAR-1) in human melanoma,” Oncogene 22, 2003, pp. 3130-3137), renal cell carcinoma (Kaufman, R., “Meizothrombin, an intermediate of prothrombin cleavage potently activates renal carcinoma cells by interaction with PAR-type thrombin receptors,” Oncology Reports; 2003, 10(2), pp. 493-496), renal disease, acute renal failure, chronic renal failure, renal vascular homeostasis (Tognetto, Michele, “Proteinase-activated receptor-1 (PAR-1) activation contracts the isolated human renal artery in vitro,” British Journal of Pharmacology, 2003, 139(1), pp. 21-27), glomerulonephritis (Ahn, Ho-Sam, “Nonpeptide thrombin receptor antagonists,” Drugs of the Future, 2001, 26(11), pp. 1065-1085), inflammation, (Meli, Rosaria, “Thrombin and PAR-1 activating peptide increase iNOS expression in cytokine-stimulated C6 glioma cells,” Journal of Neurochemistry, 2001, 79(3), pp. 556-563), chronic airways disease (Roche, Nicolas, “Effect of acute and chronic inflammatory stimuli on expression of protease-activated receptors 1 and 2 alveolar macrophages,” Journal of Allergy and Clinical Immunology, 2003, 111(2), pp. 367-373), bladder inflammation (D'Andrea, Michael R., “Expression of protease-activated receptor-1, -2, -3 and -4 in control and experimentally inflamed mouse bladder,” American Journal of Pathology, 2003, 162(3), pp. 907-923), neurodegenerative and/or neurotoxic diseases, conditions, and injuries (Traynelis, Stephen Francis, “Treatment of neurodegenerative diseases and conditions using PAR-1 antagonists,” PCT Int. Appl. WO 0271847 (2002)), radiation fibrosis, endothelial dysfunction (Wang, Junru, “Deficiency of microvascular thrombomodulin and up-regulation of protease-activated receptor-1 in irradiated rat intestine: possible link between endothelial dysfunction and chronic radiation fibrosis,” American Journal of Pathology, June 2002, 160(6), pp. 2063-72), periodontal diseases (Tanaka, Nobuhisa, “Thrombin-induced Ca²+ mobilization in human gingival fibroblasts is mediated by protease-activated receptor-1 (PAR-1),” Life Sciences, 2003, 73, pp. 301-310) and wounds (Strukova, S. M., “Thrombin, a regulator of reparation processes in wound healing,” Bioorganicheskaya Khimiya, 1998, 24(4), pp. 288-292), Thrombin receptor antagonists have also been suggested as potential antiangiogenics (Chan, Barden, “Antiangiogenic property of human thrombin,” Microvascular Research, 2003, 66(1), pp. 1-14), resistance factors for tumor cells towards chemotherapy (Schiller, H., “Thrombin as a survival factor for cancer cells: thrombin activation in malignant effusions in vivo and inhibition of idarubicin-induced cell death in vitro,” Int'l. J. of Clinical Pharmacology and Therapeutics, 2002, 40(8), pp. 329-335.), platelet aggregation inhibitors and proliferation inhibitors of smooth muscle cells, endothelial cells, fibroblasts, kidney cells, osteosarcoma cells, muscle cells, cancer cells and/or glial cells (Suzuki, supra).

Substituted thrombin receptor antagonists are disclosed in U.S. Pat. No. 6,063,847, U.S. Pat. No. 6,326,380 and U.S. Ser. Nos. 09/880222 (WO 01/96330) and 10/271715.

### SUMMARY OF THE INVENTION

In one aspect, the present invention relates to a method of treating a therapeutic condition comprising administering to a mammal in need of such treatment an effective amount of at least one compound of the formula:

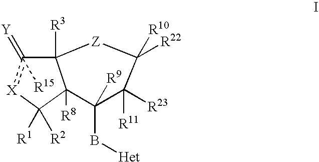

or a pharmaceutically acceptable isomer, salt, solvate or co-crystal form thereof, wherein:

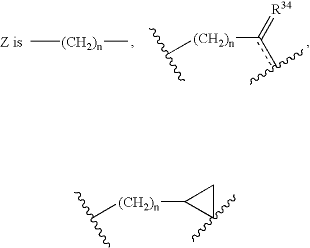

when R¹⁰ is absent, or

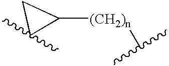

when R is absent;

the single dotted line adjacent to R³⁴ [symbol] represents an optional double bond;

the double dotted lines adjacent to X [symbol] together represent an optional single bond;

n is 0-2;

R¹ and R² are independently selected from the group consisting of H, C₁-C₆ alkyl, fluoro(C₁-C₆)alkyl, difluoro(C₁-C₆)alkyl, trifluoro-(C₁-C₆)alkyl, C₃-C₇ cycloalkyl, C₂-C₆ alkenyl, aryl(C₁-C₆)alkyl, aryl(C₂-C₆)alkenyl, heteroaryl(C₁-C₆)alkyl, heteroaryl(C₂-C₆)alkenyl, hydroxy-(C₁-C₆)alkyl, (C₁-C₆)alkoxy(C₁-C₆)alkyl, amino-(C₁-C₆)alkyl, aryl and thio(C₁-C₆)alkyl; or R¹ and R² together form a ═O group;

R³ is H, hydroxy, C₁-C₆ alkoxy, —NR¹⁸R¹⁹, —SOR¹⁶, —SO₂R¹⁷, —C(O)OR¹⁷, —C(O)NR¹⁸R¹⁹, C₁-C₆ alkyl, halogen, fluoro(C₁-C₆)alkyl, difluoro(C₁-C₆)alkyl, trifluoro(C₁-C₆)alkyl, C₃-C₇ cycloalkyl, C₂-C₆ alkenyl, aryl(C₁-C₆)alkyl, aryl(C₂-C₆)alkenyl, heteroaryl(C₁-C₆)alkyl, heteroaryl(C₂-C₆)alkenyl, hydroxy(C₁-C₆)alkyl, amino(C₁-C₆)alkyl, aryl, thio(C₁-C₆)alkyl, (C₁-C₆)alkoxy(C₁-C₆)alkyl or (C₁-C₆)alkylamino(C₁-C₆)alkyl;

R³⁴ is (H, R³), (H, R⁴³), ═O or ═NOR¹⁷ when the optional double bond adjacent to R³⁴ is absent; R³⁴ is R⁴⁴ when the double bond is present;

Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member of Het, and wherein the Het group is substituted by 1 to 4 moieties, W, independently selected from the group consisting of H; C₁-C₆ alkyl; fluoro(C₁-C₆)alkyl; difluoro(C₁-C₆)alkyl; trifluoro-(C₁-₆)-alkyl; C₃-C₇ cycloalkyl; heterocycloalkyl; heterocycloalkyl substituted by C₁-C₆ alkyl, C₂-C₆ alkenyl, OH—(C₁-C₆)alkyl, or ═O; C₂-C₆ alkenyl; R²¹-aryl(C₁-C₆)alkyl; R²¹-aryl-(C₂-C₆)-alkenyl; R²¹-aryloxy; R²¹-aryl-NH—; heteroaryl(C₁-C₆)alkyl; heteroaryl(C₂-C₆)-alkenyl; heteroaryloxy; heteroaryl-NH—; hydroxy(C₁-C₆)alkyl; dihydroxy(C₁-C₆)alkyl; amino(C₁-C₆)alkyl; (C₁-C₆)alkylamino-(C₁-C₆)alkyl; di-((C₁-C₆)alkyl)-amino(C₁-C₆)alkyl; thio(C₁-C₆)alkyl; C₁-C₆ alkoxy; C₂-C₆ alkenyloxy; halogen; —NR⁴R⁵; —CN; —OH; —COOR¹⁷; —COR¹⁶; —OSO₂CF₃; —CH₂OCH₂CF₃; (C₁-C₆)alkylthio; —C(O)NR⁴R⁵; —OCHR⁶-phenyl; phenoxy-(C₁-C₆)alkyl; —NHCOR¹⁶; —NHSO₂R¹⁶; biphenyl; —OC(R⁶)₂COOR⁷; —OC(R⁶)₂C(O)NR⁴R⁵; (C₁-C₆)alkoxy; —C(═NOR¹⁷)R¹⁸; C₁-C₆ alkoxy substituted by (C₁-C₆)alkyl, amino, —OH, COOR¹⁷, —NHCOOR¹⁷, —CONR⁴R⁵, aryl, aryl substituted by 1 to 3 moieties independently selected from the group consisting of halogen, —CF₃, C₁-C₆ alkyl, C₁-C₆ alkoxy and —COOR¹⁷, aryl wherein adjacent carbons form a ring with a methylenedioxy group, —C(O)NR⁴R⁵ or heteroaryl; R²¹-aryl; aryl wherein adjacent carbons form a ring with a methylenedioxy group; R₄₁-heteroaryl; and heteroaryl wherein adjacent carbon atoms form a ring with a C₃-C₅ alkylene group or a methylenedioxy group;

R⁴ and R⁵ are independently selected from the group consisting of H, C₁-C₆ alkyl, phenyl, benzyl and C₃-C₇ cycloalkyl, or R⁴ and R⁵ together are —(CH₂)₄—, —(CH₂)5— or —(CH₂)₂NR⁷—(CH₂)₂— and form a ring with the nitrogen to which they are attached;

R⁶ is independently selected from the group consisting of H, C₁-C₆ alkyl, phenyl, (C₃-C₇)cycloalkyl, (C₃-C₇)cycloalkyl(C₁-C₆)alkyl, (C₁-C₆)alkoxy(C₁-C₆)alkyl, hydroxy(C₁-C₆)alkyl and amino(C₁-C₆)alkyl;

R⁷ is H or (C₁-C₆)alkyl;

R⁸, R¹⁰ and R¹¹ are independently selected from the group consisting of R¹ and —OR¹, provided that when the optional double bond is present, R¹⁰ is absent;

R⁹ is H, OH, C₁-C₆ alkoxy, halogen or halo(C₁-C₆)alkyl;

B is —(CH₂)ₙ₃—, —CH₂—O—, —CH₂S—, —CH₂—NR⁶—, —C(O)NR⁶—, —NR⁶C(O)—, [symbol] cis or trans —(CH₂)ₙ₄CR¹²═CR^{12a}(CH₂)ₙ₅— or —(CH₂)ₙ₄C≡C(CH₂)ₙ₅—, wherein n₃ is 0-5, n₄ and n₅ are independently 0-2, and R¹² and R^{12a} are independently selected from the group consisting of H, C₁-C₆ alkyl and halogen;

X is —O— or —NR⁶— when the double dotted lines adjacent to X represent a single bond, or X is H, —OH or —NHR²⁰ when the bond is absent;

Y is ═O, ═S, (H, H), (H, OH) or (H, C₁-C₆ alkoxy) when the double dotted lines adjacent to X represent a single bond, or when the bond is absent, Y is ═O, ═NOR¹⁷, (H, H), (H, OH), (H, SH), (H, C₁-C₆ alkoxy) or (H, —NHR⁴⁵);

R¹⁵ is absent when the double dotted lines adjacent to X represent a single bond; R¹⁵ is H, C₁-C₆ alkyl, —NR¹⁸R¹⁹ or —OR¹⁷ when said single bond is absent; or Y is

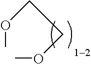

or

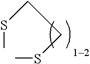

and R¹⁵ is H or C₁-C₆ alkyl;

R¹⁶ is C₁-C₆ lower alkyl, phenyl or benzyl;

R¹⁷, R¹⁸ and R¹⁹ are independently selected from the group consisting of H, C₁-C₆ alkyl, phenyl, benzyl;

R²⁰ is H, C₁-C₆ alkyl, phenyl, benzyl, —C(O)R⁶ or —SO₂R⁶;

R²¹ is 1 to 3 moieties independently selected from the group consisting of hydrogen, —CN, —CF₃, —OCF₃, halogen, —NO₂, C₁-C₆ alkyl, C₁-C₆alkoxy, (C₁-C₆)alkylamino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —NHCOR¹⁶, —NHSO₂R¹⁶, —NHSO₂CH₂CF₃, heteroaryl or —C(═NOR¹⁷)R¹⁸;

R²² and R²³ are independently selected from the group consisting of hydrogen, R²⁴—(C₁-C₁₀)alkyl, R²⁴⁻(C₂-C₁₀)alkenyl, R²⁴-(C₂-C₁₀)alkynyl, R²⁷-hetero-cycloalkyl, R²⁵-aryl, R²⁵-aryl(C₁-C₆)alkyl, R²⁹-(C₃-C₇)cycloalkyl, R²⁹-(C₃-C₇)cycloalkenyl, —OH, —OC(O)R³⁰, —C(O)OR³⁰, —C(O)R³⁰, —C(O)NR³⁰OR³¹, —NR³⁰R³¹, —NR³⁰C(O)R³¹, —NR³⁰C(O)NR³¹R³², —NHSO₂R³¹, —OC(O)NR³⁰R³¹, R²⁴—(C₁-C₁₀)alkoxy, R²⁴—(C₂-C₁₀)-alkenyloxy, R²⁴—(C₂-C₁₀)alkynyloxy, R²⁷-heterocycloalkyloxy, R²⁹—(C₃-C₇)cycloalkyloxy, R²⁹—(C₃-C₇)cyclo-alkenyloxy, R²⁹—(C₃-C₇)cycloalkyl-NH—, —CH₂—O—CH₂-phenyl, —NHSO₂NHR¹⁶ and —CH(═NOR¹⁷);

or R²² and R¹⁰ together with the carbon to which they are attached, or R²³ and R¹¹ together with the carbon to which they are attached, independently form a R⁴²-substituted carbocyclic ring of 3-10 atoms, or a R⁴²-substituted heterocyclic ring of 4-10 atoms wherein 1-3 ring members are independently selected from the group consisting of —O—, —NH— and —SO₀₋₂—, provided that when R²² and R¹⁰ form a ring, the optional double bond is absent;

R²⁴ is 1, 2 or 3 moieties independently selected from the group consisting of hydrogen, halogen, —OH, (C₁-C₆)alkoxy, R³⁵-aryl, (C₁-C₁₀)-alkyl-C(O)—, (C₂-C₁₀)-alkenyl-C(O)—, (C₂-C₁₀)alkynyl-C(O)—, heterocycloalkyl, R²⁶-(C₃-C₇)cycloalkyl, R²⁶—(C₃-C₇)cycloalkenyl, —OC(O)R³⁰, —C(O)OR³⁰, —C(O)R³⁰, —C(O)NR³⁰OR³¹, —NR³⁰R³¹, —NR³⁰C(O)R³¹, —NR³⁰C(O)NR³¹R³², —NHSO₂R³⁰, —OC(O)NR³⁰R³¹, R²⁴—(C₂-C₁₀)-alkenyloxy, R²⁴—(C₂-C₁₀)alkynyloxy, R²⁷-heterocycloalkyloxy, R²⁹—(C₃-C₇)-cycloalkyloxy, R²⁹—(C₃-C₇)cyclo-alkenyloxy, R²⁹—(C₃-C₇)cycloalkyl-NH—, —NHSO₂NHR¹⁶ and —CH(═NOR¹⁷);

R²⁵ is 1, 2 or 3 moieties independently selected from the group consisting of hydrogen, heterocycloalkyl, halogen, —COOR³, —CN, —C(O)NR³⁷R³⁸, —NR³⁹C(O)R⁴⁰, —OR³⁶, (C₃-C₇)cycloalkyl, (C₃-C₇)cycloalkyl-C₁-C₆)alkyl, (C₁-C₆)alkyl(C₃-C₇)cycloalkyl-(C₁-C₆)alkyl, halo(C₁-C₆)alkyl(C₃-C₇)cycloalkyl(C₁-C₆)alkyl, hydroxy(C₁-C₆)alkyl, (C₁-C₆)alkoxy(C₁-C₆)alkyl, and R⁴¹-heteroaryl; or two R²⁵ groups on adjacent ring carbons form a fused methylenedioxy group;

R²⁶ is 1, 2, or 3 moieties independently selected from the group consisting of hydrogen, halogen and (C₁-C₆)alkoxy;

R²⁷ is 1, 2 or 3 moieties independently selected from the group consisting of hydrogen, R²⁸—(C₁-C₁₀)alkyl, R²⁸—(C₂-C₁₀)alkenyl, R²⁸—(C₂-C₁₀)alkynyl;

R²⁸ is hydrogen, —OH or (C₁-C₆)alkoxy;

R²⁹ is 1, 2 or 3 moieties independently selected from the group consisting of hydrogen, (C₁-C₆)alkyl, —OH, (C₁-C₆)alkoxy and halogen;

R³⁰, R³¹ and R³² are independently selected from the group consisting of hydrogen, (C₁-C₁₀)-alkyl, (C₁-C₆)alkoxy(C₁-C₁₀)-alkyl, R²⁵-aryl(C₁-C₆)-alkyl, R³³—(C₃-C₇)cycloalkyl, R³⁴⁻(C₃-C₇)cycloalkyl(C₁-C₆)alkyl, R²⁵-aryl, heterocycloalkyl, heteroaryl, heterocycloalkyl(C₁-C₆)alkyl and heteroaryl(C₁-C₆)alkyl;

R³³ is hydrogen, (C₁-C₆)alkyl, OH—(C₁-C₆)alkyl or (C₁-C₆)alkoxy;

R³⁵ is 1 to 4 moieties independently selected from the group consisting of hydrogen, (C₁-C₆)alkyl, —OH, halogen, —CN, (C₁-C₆)alkoxy, trihalo(C₁-C₆)alkoxy, (C₁-C₆)alkylamino, di((C₁-C₆)alkyl)amino, —OCF₃, OH—(C₁-C₆)alkyl, —CHO, —C(O)(C₁-C₆)-alkylamino, —C(O)di((C₁-C₆)alkyl)amino, —NH₂, —NHC(O)(C₁-C₆)alkyl and —N((C₁-C₆)alkyl)C(O)(C₁-C₆)alkyl;

R³⁶ is hydrogen, (C₁-C₆)alkyl, halo(C₁-C₆)alkyl, dihalo(C₁-C₆)alkyl or trifluoro(C₁-C₆)alkyl;

R³⁷ and R³⁸ are independently selected from the group consisting of hydrogen, (C₁-C₆)alkyl, aryl(C₁-C₆)alkyl, phenyl and (C₃-C₁₅)cycloalkyl, or R³⁷ and R³⁸ together are —(CH₂)₄—, —(CH₂)₅— or —(CH₂)₂—NR³⁹—(CH₂)₂— and form a ring with the nitrogen to which they are attached;

R³⁹ and R⁴⁰ are independently selected from the group consisting of hydrogen, (C₁-C₆)alkyl, aryl(C₁-C₆)alkyl, phenyl and (C₃-C₁₅)-cycloalkyl, or R³⁹ and R⁴⁰ in the group —NR³⁹C(O)R⁴⁰, together with the carbon and nitrogen atoms to which they are attached, form a cyclic lactam having 5-8 ring members;

R⁴¹ is 1 to 4 moieties independently selected from the group consisting of hydrogen, (C₁-C₆)alkyl, (C₁-C₆)alkoxy, (C₁-C₆)alkylamino, di((C₁-C₆)alkyl)amino, —OCF₃, OH—(C₁-C₆)alkyl, —CHO and phenyl;

R⁴² is 1 to 3 moieties independently selected from the group consisting of hydrogen, —OH, (C₁-C₆)alkyl and (C₁-C₆)alkoxy;

R⁴³ is —NR³⁰R³¹, —NR³⁰C(O)R³¹, —NR³⁰C(O)NR³¹R³², —NHSO₂R³⁰ or —NHCOOR¹⁷;

R⁴⁴ is H, C₁-C₆ alkoxy, —SOR¹⁶, —SO₂R¹⁷, —C(O)OR¹⁷, —C(O)NR¹⁸R¹⁹, C₁-C₆ alkyl, halogen, fluoro(C₁-C₆)alkyl, difluoro(C₁-C₆)alkyl, trifluoro(C₁-C₆)alkyl, C₃-C₇ cycloalkyl, C₂-C₆ alkenyl, aryl(C₁-C₆)alkyl, aryl(C₂-C₆)alkenyl, heteroaryl(C₁-C₆)alkyl, heteroaryl(C₂-C₆)alkenyl, hydroxy(C₁-C₆)alkyl, amino(C₁-C₆)alkyl, aryl, thio(C₁-C₆)alkyl, (C₁-C₆)alkoxy(C₁-C₆)alkyl or (C₁-C₆)alkylamino(C₁-C₆)alkyl; and

R⁴⁵ is H, C₁-C₆ alkyl, —COOR¹⁶ or —SO₂,

wherein said therapeutic condition is a cardiovascular or circulatory disease or condition, an inflammatory disease or condition, a respiratory tract or disease or condition, cancer, acute renal failure, astrogliosis, a fibrotic disorder of the liver, kidney, lung or intestinal tract, Alzheimer's disease, diabetes, diabetic neuropathy, rheumatoid arthritis, neurodegenerative disease, neurotoxic disease, systemic lupus erythematosus, multiple sclerosis, osteoporosis, glaucoma, macular degeneration, psoriasis, radiation fibrosis, endothelial dysfunction, a wound or a spinal cord injury, or a symptom or result thereof.

In another aspect, the present invention relates to a method of treating a therapeutic condition comprising administering to a mammal in need of such treatment an effective amount of at least one compound of the formula:

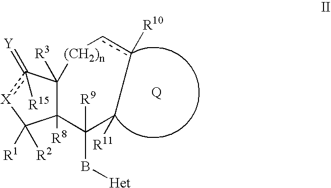

or a pharmaceutically acceptable isomer, salt, solvate or co-crystal form thereof, wherein:

the double dotted lines adjacent to X [symbol] together represent an optional single bond;

the single dotted line adjacent to R¹⁰ [symbol] represents an optional double bond;

n is 0-2;

Q is

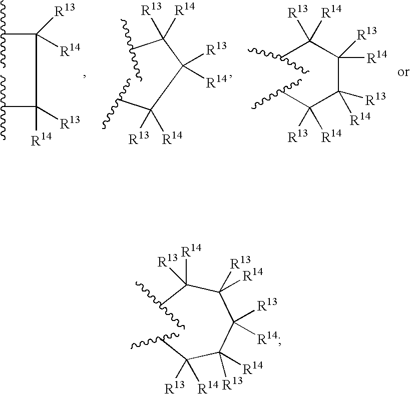

R¹ and R² are independently selected from the group consisting of H, (C₁-C₆)alkyl, fluoro(C₁-C₆)alkyl-, difluoro(C₁-C₆)alkyl-, trifluoro-(C₁-C₆)alkyl-, (C₃-C₆)cycloalkyl, (C₂-C₆)alkenyl, hydroxy-(C₁-C₆)alkyl-, and amino(C₁-C₆)alkyl-;

R³ is H, hydroxy, (C₁-C₆)alkoxy, —SOR¹⁶, —SO₂R¹⁷, —C(O)OR¹⁷, —C(O)NR¹⁸R¹⁹, —(C₁-C₆)alkyl-C(O)NR¹⁸R¹⁹, (C₁-C₆)alkyl, halogen, fluoro(C₁-C₆)alkyl-, difluoro(C₁-C₆)alkyl-, trifluoro(C₁-C₆)alkyl-, (C₃-C₆)cycloalkyl, (C₃-C₆)-cycloalkyl-(C₁-C₆)alkyl-, (C₂-C₆)alkenyl, aryl(C₁-C₆)alkyl-, aryl(C₂-C₆)alkenyl-, heteroaryl(C₁-C₆)alkyl-, heteroaryl(C₂-C₆)alkenyl-, hydroxy(C₁-C₆)-alkyl-, —NR²²R²³, NR²²R²³—(C₁-C₆)alkyl-, aryl, thio(C₁-C₆)alkyl-, (C₁-C₆)alkyl-thio(C₁-C₆)alkyl-, (C₁-C₆)alkoxy(C₁-C₆)alkyl-, NR¹⁸R¹⁹—C(O)—(C₁-C₆)alkyl- or (C₃-C₆)cycloalkyl-(C₁-C₆)alkyl-;

Het is a mono- or bi-cyclic heteroaryl group of 5 to 10 atoms comprised of 1 to 9 carbon atoms and 1 to 4 heteroatoms independently selected from the group consisting of N, O and S, wherein a ring nitrogen can form an N-oxide or a quaternary group with a (C₁-C₄)alkyl group, wherein Het is attached to B by a carbon atom ring member of said Het, and wherein the Het group is substituted by W;

W is 1 to 4 moieties independently selected from the group consisting of H, (C₁-C₆)alkyl, fluoro(C₁-C₆)alkyl-, difluoro(C₁-C₆)alkyl-, trifluoro(C₁-C₆)alkyl-, (C₃-C₆)cycloalkyl, hydroxy(C₁-C₆)alkyl-, dihydroxy(C₁-C₆)alkyl-, NR₂₅R₂₆(C₁-C₆)alkyl-, thio(C₁-C₆)alkyl-, —OH, (C₁-C₆)alkoxy, halogen, —NR⁴R⁵, —C(O)OR¹⁷, —COR¹⁶, (C₁-C₆)alkylthio-, R²¹-aryl, R²¹-aryl(C₁-C₆)alkyl-, aryl wherein adjacent ring carbons in said aryl, along with two O atoms, form a methylenedioxy group, and R²¹-heteroaryl;

R⁴ and R⁵ are independently selected from the group consisting of H, (C₁-C₆)alkyl, phenyl, benzyl and (C₃-C₆)cycloalkyl, or R⁴ and R⁵ taken together are —(CH₂)₄—, —(CH₂)₅— or —(CH₂)₂NR⁷—(CH₂)₂— and form a ring with the nitrogen to which they are attached;

R⁶ is H, (C₁-C₆)alkyl or phenyl;

R⁷ is H, (C₁-C₆)alkyl, —C(O)—R¹⁶, —C(O)OR¹⁷ or —S(O)₂R¹⁷;

R⁸, R¹⁰ and R¹¹ are independently selected from the group consisting of R¹ and —OR¹, provided that when the optional double bond shown in Formula II is present, R¹⁰ is absent;

R⁹ is H, OH or (C₁-C₆)alkoxy;

B is —(CH₂)ₙ₃—, cis or trans —(CH₂)ₙ₄CR¹²═CR^{12a}(CH₂)ₙ₅— or —(CH₂)ₙ₄C≡C(CH₂)ₙ₅—, wherein n₃ is 0-5, n₄ and n₅ are independently 0-2, and R¹² and R^{12a} are independently selected from the group consisting of H, (C₁-C₆)alkyl and halogen;

X is —O— or —NR⁶— when the dotted line shown adjacent to X in Formula II represents a single bond, or X is —OH or —NHR²⁰ when the bond is absent;

Y is ═O, ═S, (H, H), (H, OH) or (H, (C₁-C₆)alkoxy) when the dotted line shown adjacent to X in Formula II represents a single bond, or when the bond is absent, Y is ═O, (H, H), (H, OH), (H, SH) or (H, (C₁-C₆)alkoxy);

each R¹³ is independently selected from H, (C₁-C₆)alkyl, (C₃-C₈)cycloalkyl, —(CH₂)ₙ₆N HC(O)OR^{16b}, —(CH₂)ₙ₆N HC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)N²⁸R²⁹ where n₆ is 0-4, haloalkyl, and halogen;

each R¹⁴ is independently selected from H, (C₁-C₆)alkyl, —OH, (C₁-C₆)alkoxy, R²⁷-aryl(C₁-C₆)alkyl, heteroaryl, heteroarylalkyl, heterocyclyl, heterocyclylalkyl, —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4, halogen and haloalkyl; or

R¹³ and R¹⁴ taken together form a spirocyclic or a heterospirocyclic ring of 3-6 atoms;

wherein at least one of R¹³ or R¹⁴ is selected from the group consisting of —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆N HSO₂R¹⁶, —(CH₂)ₙ₆NHSO₂NR⁴R⁵, and —(CH₂)ₙ₆C(O)NR²⁸R²⁹ where n₆ is 0-4;

R¹⁵ is absent when the double dotted line shown adjacent to X in Formula II represents a single bond and is H, (C₁-C₆)alkyl, —NR¹⁸R¹⁹, or —OR¹⁷ when said bond is absent;

R¹⁶ is independently selected from the group consisting of (C₁-C₆)alkyl, phenyl and benzyl;

R^{16b} is H, alkoxy, (C₁-C₆)alkyl, (C₁-C₆)alkoxy(C₁-C₆)alkyl-, R²²—O—C(O)—(C₁-C₆)alkyl-, (C₃-C₆)cycloalkyl, R²¹-aryl, R²¹-aryl(C₁-C₆)alkyl, haloalkyl, alkenyl, halosubstituted alkenyl, alkynyl, halosubstituted alkynyl, R²¹-heteroaryl, R²¹-(C₁-C₆)alkyl heteroaryl, R²¹-(C₁-C₆)alkyl heterocycloalkyl, R²⁸R²⁹N—(C₁-C₆)alkyl, R²⁸R²⁹N—(CO)—(C₁-C₆)alkyl, R²⁸R²⁹N—(CO)O—(C₁-C₆)alkyl, R²⁸O(CO)N(R²⁹)—(C₁-C₆)alkyl, R²⁸S(O)₂N(R²⁹)—(C₁-C₆)alkyl, R²⁸R²⁹N—(CO)—N(R²⁹)—(C₁-C₆)alkyl, R²⁸R²⁹N—S(O)2N(R²⁹)—(C₁-C₆)alkyl, R²⁸—(CO)N(R²⁹)—(C₁-C₆)alkyl, R²⁸R²⁹N—S(O)₂—(C₁-C₆)alkyl, HOS(O)₂—(C₁-C₆)alkyl, (OH)₂P(O)₂—(C₁-C₆)alkyl, R²⁸—S—(C₁-C₆)alkyl, R²⁸—S(O)₂—(C₁-C₆)alkyl or hydroxy(C₁-C₆)alkyl);

R¹⁷, R¹⁸ and R¹⁹ are independently selected from the group consisting of H, (C₁-C₆)alkyl, phenyl, and benzyl;

R²⁰ is H, (C₁-C₆)alkyl, phenyl, benzyl, —C(O)R⁶ or —S(O)₂R⁶;

R²¹ is 1 to 3 moieties independently selected from the group consisting of H, —CN, —CF₃, —OCF₃, halogen, —NO₂, (C₁-C₆)alkyl, —OH, (C₁-C₆)alkoxy, (C₁-C₆)-alkylamino-, di-((C₁-C₆)alkyl)amino-, NR²⁵R²⁶⁻(C₁-C₆)alkyl-, hydroxy-(C₁-C₆)alkyl-,—C(O)OR¹⁷, —C(O)R¹⁷, —NHC(O)R¹⁶, —NHS(O)₂R¹⁶, —NHS(O)₂CH₂CF₃, —C(O)NR²⁵R²⁶, —NR²⁵—C(O)—NR²⁵R²⁶, —S(O)R¹³, —S(O)₂R¹³ and —SR¹³;

R²² is H or (C₁-C₆)alkyl;

R²³ is H, (C₁-C₆)alkyl, —C(O)R²⁴, —S(O)₂R²⁴, —C(O)NHR²⁴ or —S(O)₂NHR²⁴;

R²⁴ is (C₁-C₆)alkyl, hydroxy (C₁-C₆)alkyl or NR²⁵R²⁶—((C₁-C₆)alkyl)-;

R²⁵ and R²⁶ are independently selected from the group consisting of H and (C₁-C₆)alkyl;

R²⁷ is 1, 2 or 3 moieties selected from the group consisting of H, (C₁-C₆)alkyl, (C₃-C₆)cycloalkyl, (C₁-C₆)alkoxy, halogen and —OH; and

R²⁸ and R²⁹ are independently selected from the group consisting of H, (C₁-C₆)alkyl, (C₁-C₆)alkoxy, R²⁷-aryl(C₁-C₆)alkyl, heteroaryl, heteroarylalkyl, hydroxy(C₁-C₆)alkyl, (C₁-C₆)alkoxy(C₁-C₆)alkyl, heterocyclyl, heterocyclylalkyl, and haloalkyl; or

R²⁸ and R²⁹ taken together form a spirocyclic or a heterospirocyclic ring of 3-6 atoms,

wherein said therapeutic condition is a cardiovascular or circulatory disease or condition, an inflammatory disease or condition, a respiratory tract or disease or condition, cancer, acute renal failure, glomerulonephritis, astrogliosis, a fibrotic disorder of the liver, kidney, lung or intestinal tract, Alzheimer's disease, diabetes, diabetic neuropathy, rheumatoid arthritis, neurodegenerative disease, neurotoxic disease, systemic lupus erythematosus, multiple sclerosis, osteoporosis, glaucoma, macular degeneration, psoriasis, radiation fibrosis, endothelial dysfunction, a wound or a spinal cord injury, or a symptom or result thereof.

In yet another aspect, the present invention relates to the above methods wherein the cardiovascular or circulatory disease or condition is atherosclerosis, restenosis, hypertension, acute coronary syndrome, angina pectoris, arrhythmia, heart disease, heart failure, myocardial infarction, thrombotic or thromboembolytic stroke, a peripheral vascular disease, deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, renal vascular homeostasis or erectile dysfunction.

In yet another aspect, the present invention relates to the above methods wherein the inflammatory disease or condition is irritable bowel syndrome, Crohn's disease, nephritis or a radiation- or chemotherapy-induced proliferative or inflammatory disorder of the gastrointestinal tract, lung, urinary bladder, gastrointestinal tract or other organ.

In yet another aspect, the present invention relates to the above methods wherein the respiratory tract disease or condition is reversible airway obstruction, asthma, chronic asthma, bronchitis or chronic airways disease.

In yet another aspect, the present invention relates to the above methods wherein the cancer is renal cell carcinoma or an angiogenesis related disorder.

In yet another aspect, the present invention relates to the above methods wherein the neurodegenerative disease is Parkinson's disease, amyotropic lateral sclerosis, Alzheimer's disease, Huntington's disease or Wilson's disease.

In yet another aspect, the invention relates to a medicament for use in treating any of the above diseases or conditions comprising one or more of the compounds of Formulas I or II.

In yet another aspect, the present invention relates to the above methods further comprising administering at least one therapeutically effective agent useful in the treatment of inflammation, rheumatism, asthma, glomerulonephritis, osteoporosis, neuropathy and/or malignant tumors angiogenesis related disorders, cancer, neurodegenerative disorders, disorders of the liver, kidney and lung, melanoma, renal cell carcinoma, renal disease, acute renal failure, chronic renal failure, renal vascular homeostasis, glomerulonephritis, chronic airways disease, bladder inflammation, neurodegenerative and/or neurotoxic diseases, conditions, and injuries, radiation fibrosis, endothelial dysfunction, periodontal diseases or wounds.

In yet another aspect, the present invention relates to the above method further comprising administering at least two therapeutically effective agents.

### DETAILED DESCRIPTION

As used above, and throughout the specification, the following terms, unless otherwise indicated, shall be understood to have the following meanings:

“Subject” includes both mammals and non-mammalian animals.

“Mammal” includes humans and other mammalian animals.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties. It should be noted that any atom with unsatisfied valences in the text, schemes, examples and tables herein is assumed to have the hydrogen atom(s) to satisfy the valences.

The following definitions apply regardless of whether a term is used by itself or in combination with other terms, unless otherwise indicated. Therefore, the definition of “alkyl” applies to “alkyl” as well as the “alkyl” portions of “hydroxyalkyl”, “haloalkyl”, “alkoxy”, etc.

As used herein, the term “alkyl” means an aliphatic hydrocarbon group that can be straight or branched and comprises 1 to about 20 carbon atoms in the chain. Preferred alkyl groups comprise 1 to about 12 carbon atoms in the chain. More preferred alkyl groups comprise 1 to about 6 carbon atoms in the chain. “Branched” means that one or more lower alkyl groups such as methyl, ethyl or propyl, are attached to a linear alkyl chain. The alkyl can be substituted by one or more substituents independently selected from the group consisting of halo, aryl, cycloalkyl, cyano, hydroxy, alkoxy, alkylthio, amino, —NH(alkyl), —NH(cycloalkyl), —N(alkyl)₂ (which alkyls can be the same or different), carboxy and —C(O)O-alkyl. Non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl.

“Alkenyl” means an aliphatic hydrocarbon group (straight or branched carbon chain) comprising one or more double bonds in the chain and which can be conjugated or unconjugated. Useful alkenyl groups can comprise 2 to about 15 carbon atoms in the chain, preferably 2 to about 12 carbon atoms in the chain, and more preferably 2 to about 6 carbon atoms in the chain. The alkenyl group can be substituted by one or more substituents independently selected from the group consisting of halo, alkyl, aryl, cycloalkyl, cyano and alkoxy. Non-limiting examples of suitable alkenyl groups include ethenyl, propenyl, n-butenyl, 3-methylbut-enyl and n-pentenyl.

Where an alkyl or alkenyl chain joins two other variables and is therefore bivalent, the terms alkylene and alkenylene, respectively, are used.

“Alkoxy” means an alkyl-O-group in which the alkyl group is as previously described. Useful alkoxy groups can comprise 1 to about 12 carbon atoms, preferably 1 to about 6 carbon atoms. Non-limiting examples of suitable alkoxy groups include methoxy, ethoxy and isopropoxy. The alkyl group of the alkoxy is linked to an adjacent moiety through the ether oxygen.

“Alkynyl” means an aliphatic hydrocarbon group comprising at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. Non-limiting examples of suitable alkynyl groups include ethynyl, propynyl, 2-butynyl, 3-methylbutynyl, n-pentynyl, and decynyl. The alkynyl group may be substituted by one or more substituents which may be the same or different, each substituent being independently selected from the group consisting of alkyl, aryl and cycloalkyl.

“Aryl” means an aromatic monocyclic or multicyclic ring system comprising about 5 to about 14 carbon atoms, preferably about 6 to about 10 carbon atoms. The aryl can be substituted with one or more substituents, as defined above, which may be the same or different. Non-limiting examples of suitable aryl groups include phenyl, naphthyl, indenyl, tetrahydronaphthyl and indanyl. “Arylene” means a bivalent phenyl group, including ortho, meta and para-substitution.

Substitution on alkyl, alkenyl and alkynyl chains depends on the length of the chain, and the size and nature of the substituent. Those skilled in the art will appreciate that while longer chains can accommodate multiple substituents, shorter alkyl chains, e.g., methyl or ethyl, can have multiple substitution by halogen, but otherwise are likely to have only one or two substituents other than hydrogen. Shorter unsaturated chains, e.g., ethenyl or ethynyl, are generally unsubstituted or substitution is limited to one or two groups, depending on the number of available carbon bonds.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be substituted with one or more substituents, as defined above, which may be the same or different. Non-limiting examples of suitable monocyclic cycloalkyls include cyclopropyl, cyclobutyl, cyclopentyl, cyclohexyl and the like. Non-limiting examples of suitable multicyclic cycloalkyls include 1-decalinyl, norbornyl, adamantyl and the like. “Cycloalkylene” refers to a corresponding bivalent ring, wherein the points of attachment to other groups include all positional isomers.

“Dihydroxy(C₁-C₆)alkyl” refers to an alkyl chain substituted by two hydroxy groups on two different carbon atoms.

“Fluoroalkyl”, “difluoroalkyl” and “trifluoroalkyl” mean alkyl chains wherein the terminal carbon is substituted by 1, 2 or 3 fluoroatoms, respectively, e.g., —CF₃, —CH₂CF₃, —CH₂CHF₂ or —CH₂CH₂F. “Haloalkyl” means an alkyl chain substituted by 1 to 3 halo atoms.

“Halogen” or “halo” refers to fluorine, chlorine, bromine or iodine radicals. Preferred are fluoro, chloro or bromo, and more preferred are fluoro and chloro.

“Heteroaryl” means an aromatic monocyclic or multicyclic ring system comprising 5 to 14 ring atoms, preferably about 5 to 10 ring atoms, comprised of 1 to 13 carbon atoms and 1 to 4 heteroatoms independently selected from the group consisting of N, O and S, provided that the rings do not include adjacent oxygen and/or sulfur atoms. N-oxides of the ring nitrogens are also included, as well as compounds wherein a ring nitrogen is substituted by a (C₁-C₄)alkyl group to form a quaternary amine. Examples of single-ring heteroaryl groups are pyridyl, oxazolyl, isoxazolyl, oxadiazolyl, furanyl, pyrrolyl, thienyl, imidazolyl, pyrazolyl, tetrazolyl, thiazolyl, isothiazolyl, thiadiazolyl, pyrazinyl, pyrimidyl, pyridazinyl and triazolyl. Examples of bicyclic heteroaryl groups are naphthyridyl (e.g., 1, 5 or 1,7), imidazopyridyl, pyrido[2,3]imidazolyl, pyridopyrimidinyl and 7-azaindolyl. Examples of benzofused heteroaryl groups are indolyl, quinolyl, isoquinolyl, phthalazinyl, benzothienyl (i.e., thionaphthenyl), benzimidazolyl, benzofuranyl, benzoxazolyl and benzofurazanyl. All positional isomers are contemplated, e.g., 2-pyridyl, 3-pyridyl and 4-pyridyl. W-substituted heteroaryl refers to such groups wherein substitutable ring carbon atoms have a substituent as defined above, or where adjacent carbon atoms form a ring with an alkylene group or a methylenedioxy group, or where a nitrogen in the Het ring can be substituted with R²¹-aryl or an optionally substituted alkyl substituent as defined in W.

The term “Het” is exemplified by the single ring, the ring substituted with another ring (which can be the same or different), benzofused heteroaryl groups as defined immediately above, as well as tricyclic groups such as benzoquinolinyl (e.g., 1,4 or 7,8) or phenanthrolinyl (e.g., 1,7; 1,10; or 4,7). Het groups are joined to group B by a carbon ring member, e.g., Het is 2-pyridyl, 3-pyridyl or 2-quinolyl.

Examples of heteroaryl groups wherein adjacent carbon atoms form a ring with an alkylene group are 2,3-cyclopentenopyridine, 2,3-cyclohexenopyridine and 2,3-cycloheptenopyridine.

“Heterocycloalkyl” means a 4 to 6 membered saturated ring containing 3 to 5 carbon atoms and 1 or 2 heteroatoms selected from the group consisting of N, S and O, provided that the heteroatoms are not adjacent. Examples of heterocycloalkyl rings are pyrrolidinyl, piperidinyl, piperazinyl, morpholinyl, thiomorpholinyl, thiazolidinyl, 1,3-dioxolanyl, 1,4-dioxanyl, tetrahydrofuranyl, tetrahydrothiophenyl and tetrahydrothiopyranyl.

The term “heterospirocyclic” refers to a spirocyclic structure containing 3 to 5 carbon atoms and 1 or 2 heteroatoms selected from the group consisting of N, S and O, provided that the heteroatoms are not adjacent.

The term “optional single bond” represented by [symbol] refers to the bond shown by the double dotted line between X and the carbon to which Y and R¹⁵ are attached in the structures of Formulas I and II. “Optional single bond” means that a single bond may be present, or that no bond is present. The “optional double bond” represented by [symbol] refers to the bond shown by the combined solid/single dotted line in the middle ring of the structure shown for Formulas I and II and means that at least a single bond must be present, but that a double bond can be present. When the double bond is present, R¹⁰ is absent.

When R⁴ and R⁵ join to form a ring with the nitrogen to which they are attached, the rings formed are 1-pyrrolidinyl, 1-piperidinyl and 1-piperazinyl, wherein the piperazinyl ring may also be substituted at the 4-position nitrogen by a group R⁷.

The above statements, wherein, for example, R⁴ and R⁵ are said to be independently selected from a group of substituents, means that R⁴ and R⁵ are independently selected when attached to the same nitrogen, but also that where an R⁴ or R⁵ variable occurs more than once in a molecule, those occurrences are independently selected. Similarly, each occurrence of R¹³ or R¹⁴ is independent of any other R¹³ or R¹⁴ in the same Q ring. Those skilled in the art will recognize that the size and nature of the substituent(s) will affect the number of substituents which can be present.

Compounds of the invention have at least one asymmetrical carbon atom and therefore all isomers, including enantiomers, stereoisomers, rotamers, tautomers and racemates of the compounds of Formula I or II (where they exist) are contemplated as being part of this invention. The invention includes d and l isomers in both pure form and in admixture, including racemic mixtures. Isomers can be prepared using conventional techniques, either by reacting optically pure or optically enriched starting materials or by separating isomers of a compound of Formula I or II. Isomers may also include geometric isomers, e.g., when a double bond is present.

“Polymorph” means a crystalline form of a substance that is distinct from another crystalline form but that shares the same chemical formula. Polymorphous forms of the compounds of Formula I or II, whether crystalline or amorphous, also are contemplated as being part of this invention.

It should also be noted that any formula, compound, moiety or chemical illustration with unsatisfied valences in the present specification and/or claims herein is assumed to have sufficient hydrogen atom(s) to satisfy the valences.

“Effective amount” or “therapeutically effective amount” is meant to describe an amount of compound or a composition of the present invention effective in antagonism of a thrombin receptor and thus producing the desired therapeutic, ameliorative, inhibitory or preventative effect.

Those skilled in the art will appreciate that for some of the compounds of Formula I or II, one isomer will show greater pharmacological activity than other isomers.

Typical preferred compounds of Formulas I and II have the following stereochemistries:

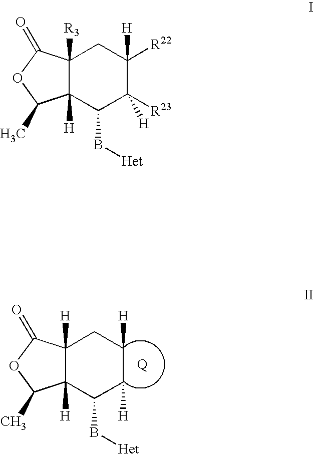

with compounds having these absolute stereochemistries being more preferred.

Compounds of the invention with a basic group can form pharmaceutically acceptable salts with organic and inorganic acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. Preferred embodiments include bisulfate salts. The salt is prepared by contacting the free base form with a sufficient amount of the desired acid to produce a salt. The free base form may be regenerated by treating the salt with a suitable dilute aqueous base solution such as dilute aqueous sodium bicarbonate. The free base form differs from its respective salt form somewhat in certain physical properties, such as solubility in polar solvents, but the salt is otherwise equivalent to its respective free base forms for purposes of the invention. Compounds of the invention can also form pharmaceutically acceptable solvates, including hydrates.

Certain compounds of the invention are acidic (e.g., those compounds which possess a carboxyl group). These compounds form pharmaceutically acceptable salts with inorganic and organic bases. Examples of such salts are the sodium, potassium, calcium, aluminum, lithium, gold and silver salts. Also included are salts formed with pharmaceutically acceptable amines such as ammonia, alkyl amines, hydroxyalkylamines, N-methylglucamine and the like.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of Formula I or II or a salt and/or solvate thereof (e.g., a prodrug on being brought to the physiological pH or through enzyme action is converted to the desired drug form). A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Co-crystal” means a crystalline structure simultaneously comprising pharmaceutically active molecules and inert molecules. Co-crystals may be formed by combining a weak base with a weak acid selected to match hydrogen bond donors with acceptors. The pKa difference of conjugate pairs may be inconsistent with salt formation in water. The co-crystallizing agents used to form co-crystals are usually bifunctional acids such as fumaric acid, succinic acid, malic acid, and tartaric acid. Co-crystals are discussed in J. F. Remenar et. al., “Crystal Engineering of Novel Cocrystals of a Triazole Drug with 1,4-Dicarboxylic Acids”, Journal of the American Chemical Society, 2003, vol.125, pp. 8456-8457.

Compounds of the invention with a carboxylic acid group can form pharmaceutically acceptable esters with an alcohol. Examples of suitable alcohols include methanol and ethanol.

Compounds of Formulas I and II are prepared by processes described with synthetic schemes and preparative examples disclosed in U.S. Pat. No. 6,645,987 and application Ser. No. 10/412,982, respectively, which schemes and examples are incorporated by reference herein.

Compounds of Formula I

For compounds of Formula I, preferred definitions of the variables are as follows:

R², R⁸, R¹⁰ and R¹¹ are each preferably hydrogen. R³ preferably is hydrogen, OH, C₁-C₆ alkoxy, —NHR¹⁸ or C₁-C₆ alkyl. The variable n is preferably zero or one. R⁹ is preferably H, OH or alkoxy. R¹ is preferably C₁-C₆ alkyl, more preferably methyl. The double dotted line preferably represents a single bond; X is preferably —O— and Y is preferably ═O or (H, —OH). B is preferably trans —CH═CH—. Het is preferably pyridyl, substituted pyridyl, quinolyl or substituted quinolyl. Preferred substituents (W) on Het are R²¹-aryl, R⁴¹-heteroaryl or alkyl. More preferred are compounds wherein Het is 2-pyridyl substituted in the 5-position by R²¹-aryl, R⁴¹-heteroaryl or alkyl, or 2-pyridyl substituted in the 6-position by alkyl. R³⁴ is preferably (H,H) or (H,OH).

R²² and R²³ are preferably selected from OH, (C₁-C₁₀)alkyl, (C₂-C₁₀)-alkenyl, (C₂-C₁₀)-alkynyl, trifluoro(C₁-C₁₀)alkyl, trifluoro(C₂-C₁₀)-alkenyl, trifluoro(C₂-C₁₀)alkynyl, (C₃-C₇)-cycloalkyl, R²⁵-aryl, R²⁵-aryl(C₁-C₆)alkyl, R²⁵-arylhydroxy(C₁-C₆)alkyl, R²⁵-aryl-alkoxy-(C₁-C₆)alkyl, (C₃-C₇)cycloalkyl-(C₁-C₆)alkyl, (C₁-C₁₀)alkoxy, (C₃-C₇)cycloalkyloxy, (C₁-C₆)alkoxy(C₁-C₆)alkyl, OH—(C₁-C₆)alkyl, trifluoro(C₁-C₁₀)alkoxy and R²⁷-heterocyclo-alkyl(C₁-C₆)alkyl. More preferred are compounds wherein R²² and R²³ are independently selected from the group consisting of (C₁-C₁₀)alkyl and OH—(C₁-C₆)alkyl.

More preferably, the present invention relates to thrombin receptor antagonists represented by any of the following structural formulas:

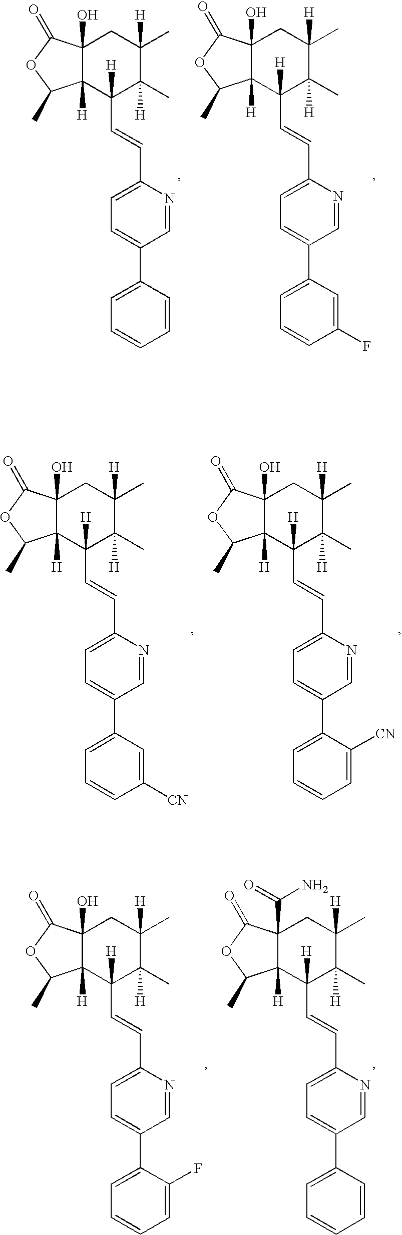

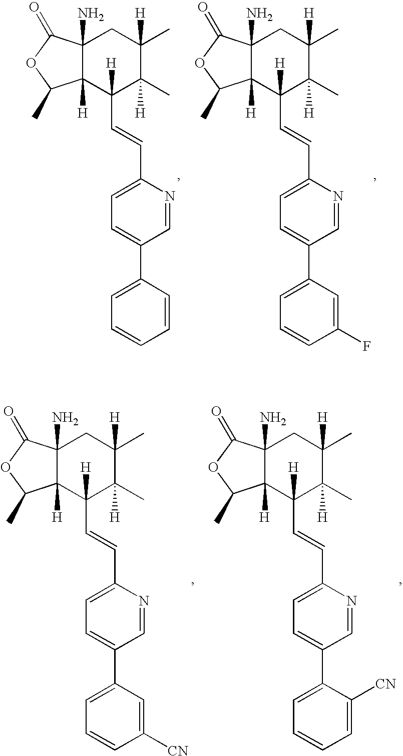

or a pharmaceutically acceptable isomer, salt, solvate, polymorph or co-crystal thereof.

Following are examples of compounds of Formula I.

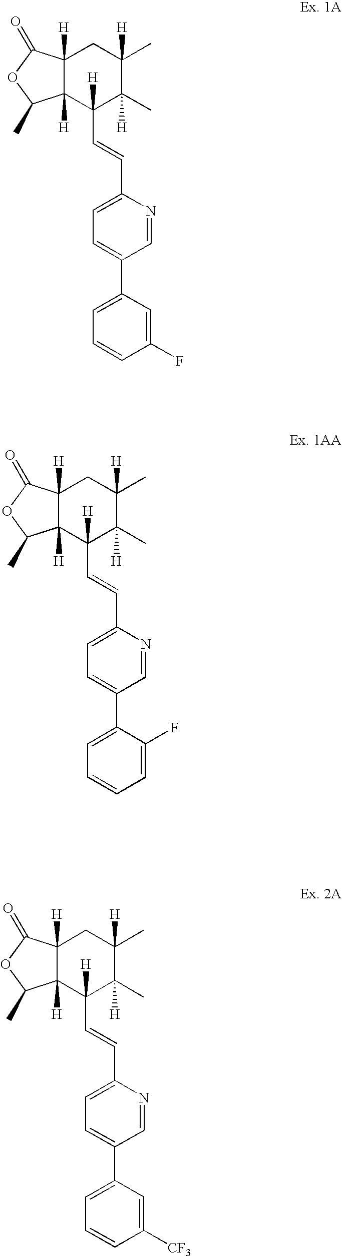

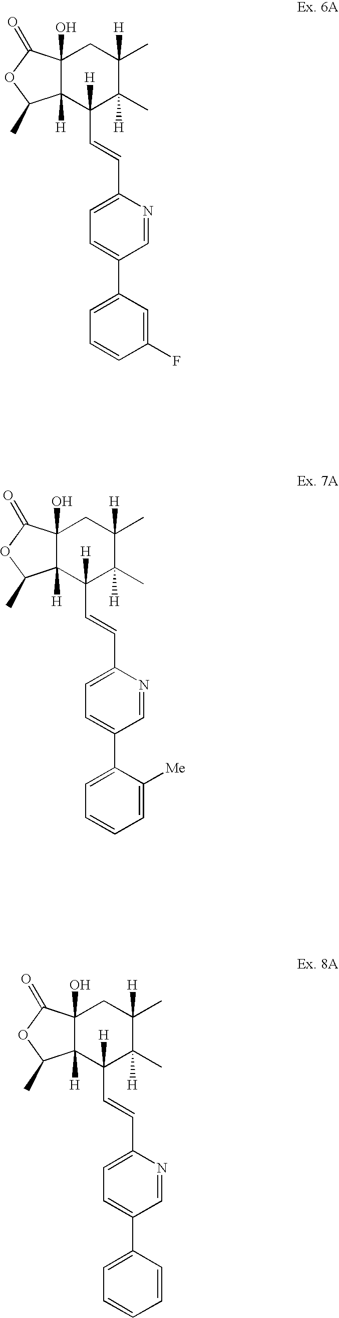

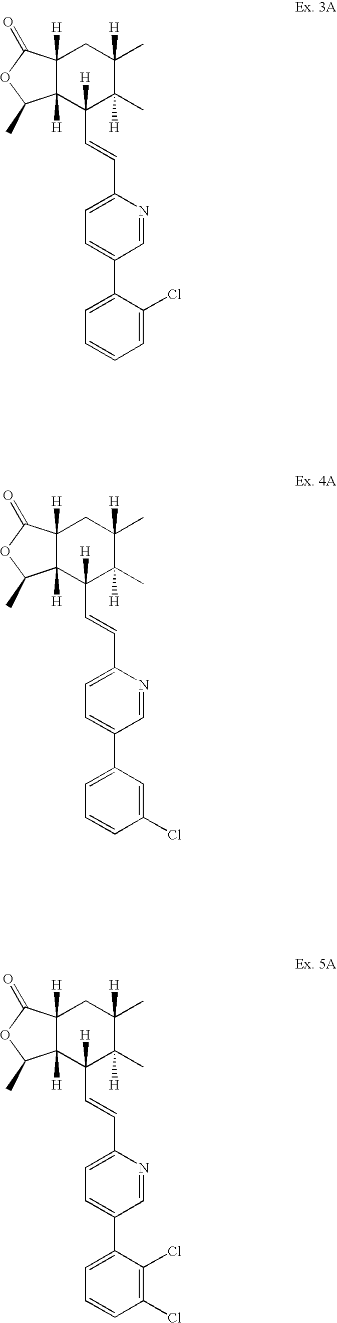

Still further compounds of Example 8 are disclosed in Table 1.

TABLE 1

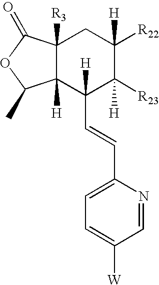

| | | | | | Analytical |
|---|---|---|---|---|---|
| Ex. | R³ | R²² | R²³ | W | Data |
| 8AA | H | Me | Et | 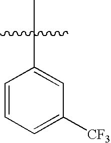 | HRMS (MH⁺) 444.2165 |
| 8BA | H | Me | Et | 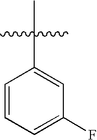 | HRMS (MH⁺) 394.2184 |
| 8CA | H | Me | Et | 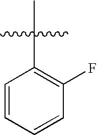 | HRMS (MH⁺) 394.2184 |
| 8DA | H | Me | Et | 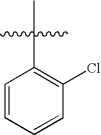 | HRMS (MH⁺) 410.1891 |
| 8EA | H | Me | Et | 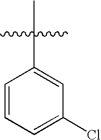 | HRMS (MH⁺) 410.1887 |
| 8FA | H | Me | Et | 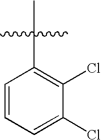 | HRMS (MH⁺) 444.1491 |
| 8GA | H | H | Ph | 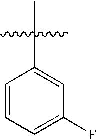 | HRMS (MH⁺) 428.2026 |
| 8HA | H | H | Ph | 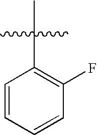 | HRMS (MH⁺) 428.2027 |
| 8IA | H | Me | Et | 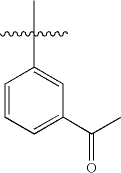 | HRMS (MH⁺) 418.2381 |
| 8JA | H | Me | Et | 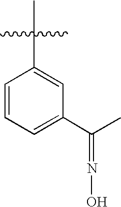 | HRMS (MH⁺) 433.2490 |
| 8KA | H | Me | Et | 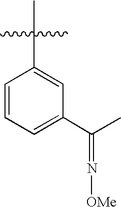 | HRMS (MH⁺) 447.2648 |
| 8LA | H | Me | Et | 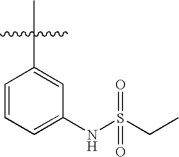 | HRMS (MH⁺) 483.2319 |
| 8MA | H | Me | Et | 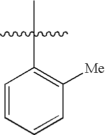 | HRMS (MH⁺) 390.2441 |
| 8NA | H | Me | Et | 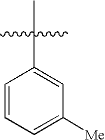 | HRMS (MH⁺) 390.2437 |
| 8OA | H | Me | Et | 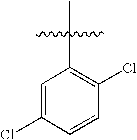 | HRMS (MH⁺) 444.1490 |
| 8PA | Me | Me | Et | 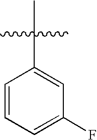 | HRMS (MH⁺) 408.2346 |
| 8QA | OH | Me | Et | 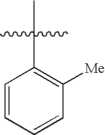 | HRMS (MH⁺) 406.2380 |
| 8RA | OH | Me | Et | 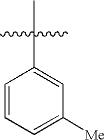 | HRMS (MH⁺) 406.2376 |
| 8SA | OH | Me | Et | 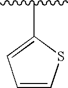 | HRMS (MH⁺) 398.1788 |
| 8TA | OH | Me | Et | 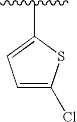 | HRMS (MH⁺) 432.1392 |
| 8UA | OH | Me | Et | 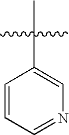 | HRMS (MH⁺) 393.2181 |
| 8VA | OH | Me | Et | 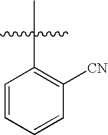 | HRMS (MH⁺) 417.2178 |
| 8WA | OH | Me | Et | 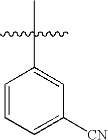 | HRMS (MH⁺) 417.2178 |
| 8XA | OH | Me | Et | 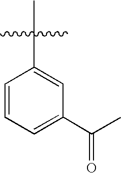 | HRMS (MH⁺) 434.2330 |
| 8YA | OH | Me | Et | 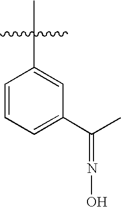 | HRMS (MH⁺) 449.2440 |
| 8ZAA | OH | Me | Et | 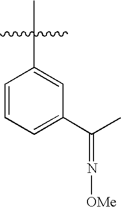 | HRMS (MH⁺) 463.2599 |
| 8AAA | OH | Me | Et | 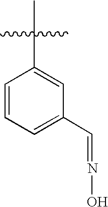 | HRMS (MH⁺) 435.2275 |
| 8ABA | OH | Me | Et | 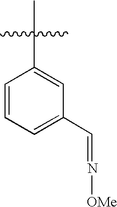 | HRMS (MH⁺) 449.2446 |
| 8ACA | OH | Me | Et | 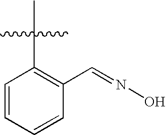 | HRMS (MH⁺) 435.2279 |
| 8ADA | OH | Me | Et | 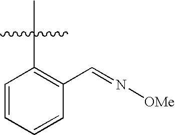 | HRMS (MH⁺) 449.2442 |
| 8AEA | OH | Me | Et | 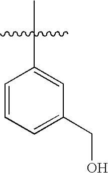 | HRMS (MH⁺) 422.2332 |
| 8AFA | OH | Me | Et | 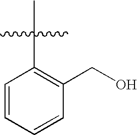 | HRMS (MH⁺) 422.2332 |
| 8AGA | H | H | Et | 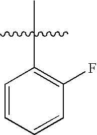 | HRMS (MH⁺) 380.2028 |
| 8AHA | H | Ph | Me | 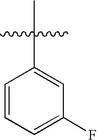 | MS (MH⁺) 442.1 |
| 8AIA | H | Ph | Me | 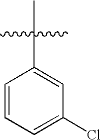 | MS (MH⁺) 458.1 |
| 8AJA | OH | Me | Et | 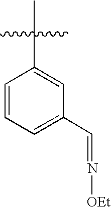 | HRMS (MH⁺) 463.2589 |
| 8AKA | OH | Me | Et | 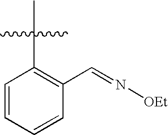 | HRMS (MH⁺) 463.2593 |
| 8ALA | OH | Me | Et | 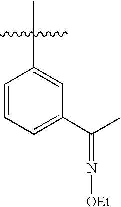 | HRMS (MH⁺) 477.2750 |
| 8AMA | OH | Me | Et | 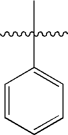 | HRMS (MH⁺) 392.2227 |
| 8ANA | OH | Me | Et | 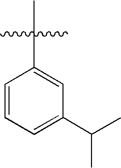 | HRMS (MH⁺) 434.2695 |
| 8AOA | OH | Me | Et | 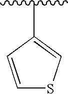 | HRMS (MH⁺) 398.1788 |
| 8APA | OH | Me | Et | 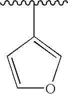 | HRMS (MH⁺) 382.2020 |
| 8AQA | OH | Me | Et | 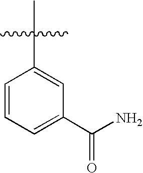 | HRMS (MH⁺) 435.2282 |
| 8ARA | OH | Me | Et | 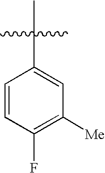 | HRMS (MH⁺) 424.0945 |
| 8ASA | OMe | Me | Et | 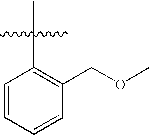 | MS (MH⁺) 450.1 |
| 8ATA | OH | Me | Et | 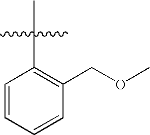 | MS (MH⁺) 436.1 |
| 8AUA | OMe | Me | Et | 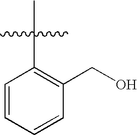 | MS (MH⁺) 436.1 |
| 8AVA | OH | Me | Et | 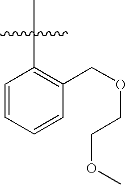 | HRMS (MH⁺) 480.2752 |
| BAWA | OH | Me | Et | 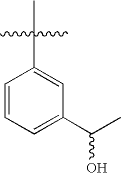 | HRMS (MH⁺) 436.2489 |
| 8AXA | OH | Me | Et | 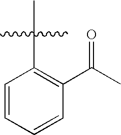 | HRMS (MH⁺) 434.2325 |
| 8AYA | OH | Me | Et | 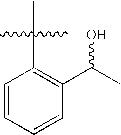 | HRMS (MH⁺) 436.2489 |
| 8AZA | OH | H | Et | 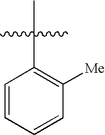 | MS (MH⁺) 392.2 |
| 8BAA | OH | H | Et | 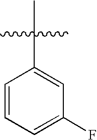 | MS (MH⁺) 396.3 |
| 8BBA | OH | H | Et | 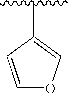 | MS (MH⁺) 368.4 |
| 8BCA | OH | Me | Et | 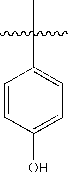 | HRMS (MH⁺) 408.2169 |
| 8BDA | OH | Me | Et | 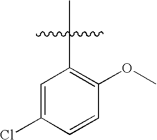 | HRMS (MH⁺) 456.1941 |
| 8BEA | OH | H | Me | 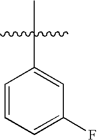 | HRMS (MH⁺) 382.1813 |
| 8BFA | OH | H | Me | 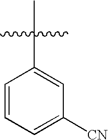 | HRMS (MH⁺) 389.1863 |
| 8BGA | OH | H | Me | 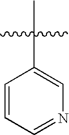 | HRMS (MH⁺) 365.1871 |
| 8BHA | OH | Me | Et | 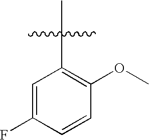 | HRMS (MH⁺) 440.2243 |
| 8BIA | OH | H | Me | 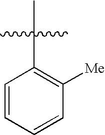 | HRMS (MH⁺) 378.2064 |
| 8BJA | OH | H | Me | 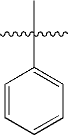 | HRMS (MH⁺) 364.1919 |
| 8BKA | OH | Me | Et | 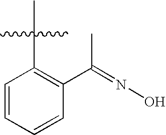 | HRMS (MH⁺) 449.2435 |
| 8BLA | OH | Me | Et | 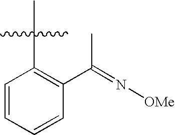 | HRMS (MH⁺) 463.2604 |
| 8BMA | OH | Me | Et | 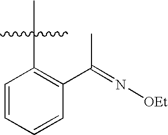 | HRMS (MH⁺) 477.2751 |
| 8BNA | OH | Me | Et | 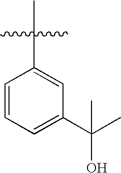 | HRMS (MH⁺) 450.2640 |

Still further compounds of Example 8 are disclosed in Table 2.

TABLE 2

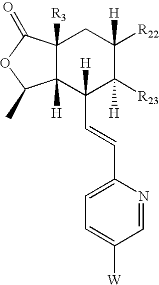

| | | | | | Analytical |
|---|---|---|---|---|---|
| Ex. | R₃ | R₂₂ | R₂₃ | W | Data |
| 8BPA | OH | H | Me | 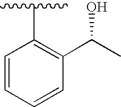 | HRMS (MH⁺) 408.2181 |
| 8BQA | OH | H | Me | 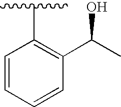 | HRMS (MH⁺) 408.2181 |
| 8BRA | OH | Me | Et | 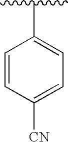 | HRMS (MH⁺) 417.2182 |
| 8BSA | OH | H | Me | 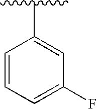 | HRMS (MH⁺) 366.1867 |
| 8BTA | OH | Me | Et | 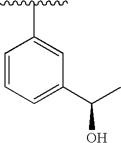 | HRMS (MH⁺) 436.2493 |
| 8BUA | OH | Me | Me | 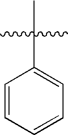 | HRMS (MH⁺) 378.2075 |
| 8BVA | OH | H | Me | 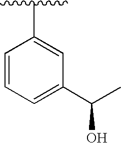 | HRMS (MH⁺) 408.2173 |
| 8BWA | OH | H | Me | 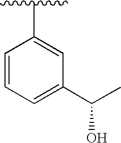 | HRMS (MH⁺) 408.2169 |
| 8BXA | OH | Me | Et | 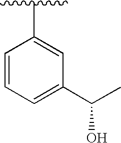 | HRMS (MH⁺) 436.2492 |
| 8BYA | OH | Me | Me | 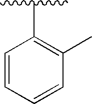 | HRMS (MH⁺) 392.2231 |
| 8BZA | H | Me | Et | 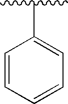 | MS (MH⁺) 376.1 |
| 8CAA | OH | Me | Me | 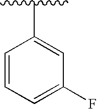 | HRMS (MH⁺) 396.1969 |
| 8CBA | OH | Me | Me | 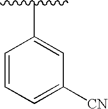 | MS (MH⁺) 403.1 |
| 8CCA | OH | Me | Me | 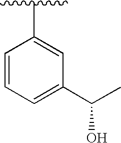 | HRMS (MH⁺) 422.2337 |
| 8CDA | OH | Me | Et | 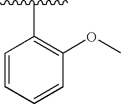 | HRMS (MH⁺) 422.2336 |
| 8CEA | OH | Me | Et | 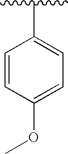 | HRMS (MH⁺) 422.2331 |
| 8CFA | OH | Me | Et | 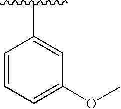 | HRMS (MH⁺) 422.2336 |
| 8CGA | OH | Me | Et | 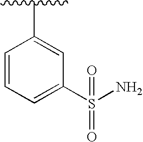 | HRMS (MH⁺) 471.1961 |
| 8CHA | OH | Me | Et | 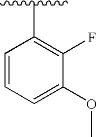 | HRMS (MH⁺) 440.2234 |
| 8CIA | OH | Me | Et | 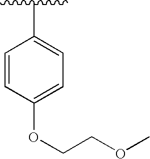 | HRMS (MH⁺) 466.2600 |
| 8CJA | OH | Me | Me | 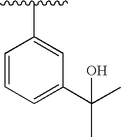 | MS (MH⁺) 436.1 |
| 8CKA | OH | Me | Me | 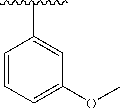 | MS (MH⁺) 409.1 |
| 8CLA | OH | Me | Me | 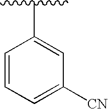 | HRMS (MH⁺) 403.2027 |
| 8CMA | OH | Me | Me | 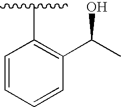 | HRMS (MH⁺) 422.2336 |
| 8CNA | OH | Me | Me | 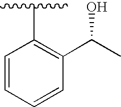 | MS (MH⁺) 422.1 |
| 8COA | H | Et | Et | 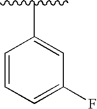 | MS (MH⁺) 408.1 |
| 8CPA | H | Me | Et | 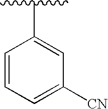 | MS (MH⁺) 401.1 |
| 8CQA | OH | Et | Et | 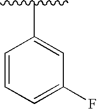 | MS (MH⁺) 424.1 |
| 8CRA | H | Me | Me | 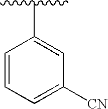 | MS (MH⁺) 387.1 |
| 8CSA | H | Me | Me | 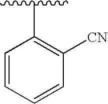 | MS (MH⁺) 387.1 |
| 8CTA | H | Et | Et | 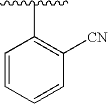 | MS (MH⁺) 415.1 |
| 8CUA | OH | Me | Me | 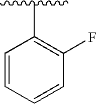 | MS (MH⁺) 396.2 |

### EXAMPLE 9A

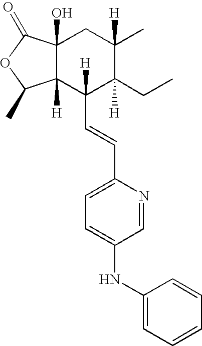

Similar compounds of the formula

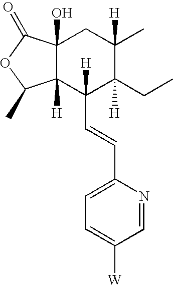

were prepared, wherein W is as defined Table 3:

TABLE 3

| | | Analytical |
|---|---|---|
| Ex. | W | Data |
| 9AA | 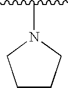 | HRMS (MH⁺) 385.2490 |
| 9BA | 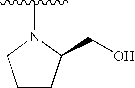 | HRMS (MH⁺) 415.2601 |
| 9CA | 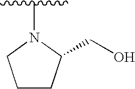 | HRMS (MH⁺) 414.2593 |
| 9DA | 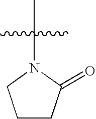 | HRMS (MH⁺) 399.2278 |

### EXAMPLE 10A

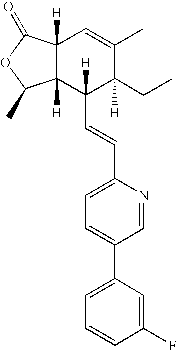

Compounds of Formula II

For compounds of Formula II, preferred definitions of the variables are as follows:

The variable n is preferably 0-2, and more preferably 0. The optional double bond is preferably absent (i.e., the bond is a single bond).

Q is preferably:

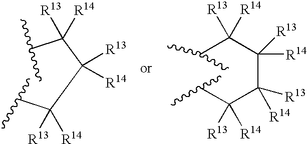

with the six-membered Q ring being more preferred. R¹³ is preferably H or —CH₃. R¹⁴ is preferably H or —CH₃. For the five-membered Q ring, preferably no more than two R¹³ and R¹⁴ substituents are other than hydrogen. For the six-membered Q ring, preferably no more than four R¹³ and R¹⁴ substituents are other than hydrogen, more preferably no more than two R¹³ and R¹⁴ substituents are other than hydrogen.

Especially preferred Q rings are:

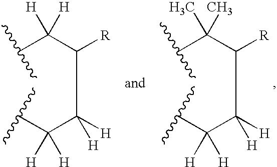

preferably shown as

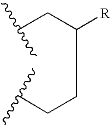

and

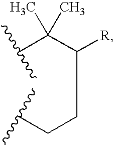

respectively.

In the preferred Q rings above, R is preferably —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶ or —(CH₂)ₙ₆NHSO₂NR⁴R⁵ wherein n₆ is 0-2, and R^{16b}, R¹⁶ and R⁴ are (C₁-C₆)alkyl and R⁵ is H. More preferred are compounds of Formula II wherein R is —NHC(O)OR^{16b}, —NHC(O)R^{16b}, —NHC(O)NR⁴R⁵, —NHSO₂R¹⁶ or —NHSO₂NR⁴R⁵ wherein R^{16b}, R¹⁶ and R⁴ are (C₁-C₆)alkyl and R⁵ is H. Even more preferred are compounds of Formula II wherein R is —NHC(O)OR^{16b}, —NHC(O)R^{16b} or —NHC(O)NR⁴R⁵, wherein R^{16b} and R⁴ are (C₁-C₆)alkyl and R⁵ is H.

R¹ and R² are preferably independently selected from the group consisting of H and (C₁-C₆)alkyl; more preferably, R¹ is (C₁-C₆)alkyl and R² is H; especially preferred are compounds wherein R¹ is —CH₃ and R² is H.

R³ is preferably H, —OH, (C₁-C₆)alkyl, (C₁-C₆)alkoxy, halogen, (C₃-C₆)cycloalkyl, —C(O)OR₇ or —NR²²R²³; more preferably, R³ is H or (C₁-C₆)alkyl.

Het is preferably pyridyl attached to B by a carbon ring member, and is preferably substituted by 1 or 2 substituents selected from W, more preferably 1 substituent. W is preferably R²¹-aryl or R²¹-heteroaryl. Aryl is preferably phenyl. Heteroaryl is preferably pyridyl. R²¹ is preferably H, halogen or —CN, or —CF₃, especially F, —CN or —CF₃.

R⁸, R¹⁰ and R¹¹ are each independently preferably H or (C₁-C₆)alkyl, more preferably H or —CH₃; especially preferred are compounds of Formula II wherein R⁸, R¹⁰ and R¹¹ are each H.

R⁹ is preferably H, OH or (C₁-C₆)alkoxy; more preferably, R⁹ is H.

B is preferably cis or trans —(CH₂)ₙ₄CR¹²═CR^{12a}(CH₂)ₙ₅— wherein n₄, n₅, R¹² and R^{12a} are as defined above; more preferably, R¹² and R^{12a} are each H, and n₄ and n₅ are each zero. Particularly preferred are compounds wherein B is trans-alkenyl, especially —CH═CH—.

One group of preferred compounds is that wherein the optional single bond is present, X is —O—, Y is ═O, and R¹⁵ is absent. Another preferred group of compounds is that wherein the optional single bond is absent, X is —OH, Y is (H,OH) and R¹⁵ is H. Compounds wherein the optional single bond is present, X is —O—, Y is ═O, and R¹⁵ is absent are more preferred.

Especially preferred are compounds of Formula II wherein R is —NHC(O)OR^{16b} wherein R^{16b} is (C₁-C₆)alkyl. R_{16b} is preferably methyl or ethyl. Also preferred are compounds wherein the R group is attached to the C-7 position of the Q ring, as shown in Formula IIAB below.

A preferred embodiment of the invention is a compound of Formula IIAB:

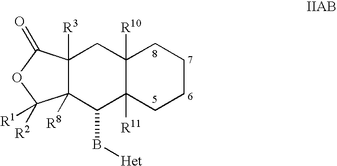

wherein R¹, R², R³, R⁸, R¹⁰, R¹¹, B, and Het are defined as preferred above. At least one of ring carbon atoms 5-8 is preferably substituted with —(CH₂)ₙ₆NHC(O)OR^{16b}, —(CH₂)ₙ₆NHCOR^{16b}, —(CH₂)ₙ₆NHCONR⁴R⁵, —(CH₂)ₙ₆NHSO₂R¹⁶ or —(CH₂)ₙ₆NHSO₂NR⁴R⁵ wherein n₆ is 0-2, and R^{16b}, R¹⁶ and R⁴ are (C₁-C₆)alkyl and R⁵ is H.

A more preferred embodiment of the invention is a compound of Formula IIBB:

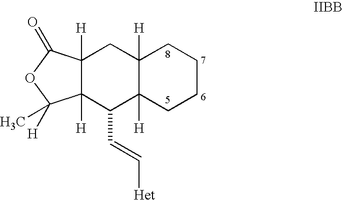

wherein Het is pyridyl substituted by an R²¹-aryl group, preferably an R²¹-phenyl group wherein R²¹ is preferably F, —CN or —CF₃.

Especially preferred are compounds of Formula IIAB or IIBB wherein at least one of ring carbon atoms 5-8 is substituted with —NHC(O)OR^{16b} wherein R^{16b} is (C₁-C₆)alkyl. R^{16b} is preferably methyl or ethyl.

Compounds of Formula II in which n₆ is 0 can be prepared by processes known in the art, for example by the processes described in U.S. Pat. No. 6,063,847, incorporated herein by reference.

Compounds of Formula II in which n₆ is 1 or 2 are generally prepared by processes in accordance with the schemes disclosed in U.S. application Ser. No. 10/412,982.

Following are examples of compounds of Formula II.

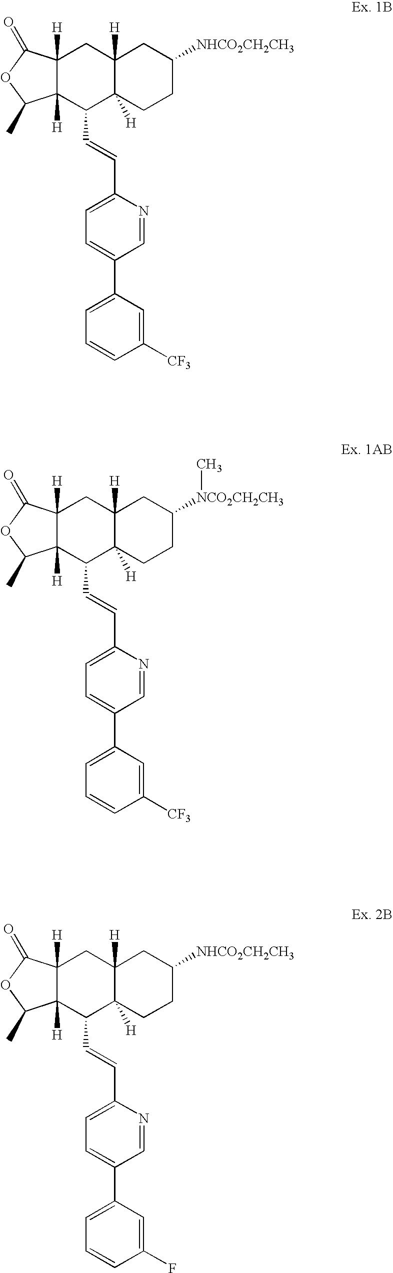

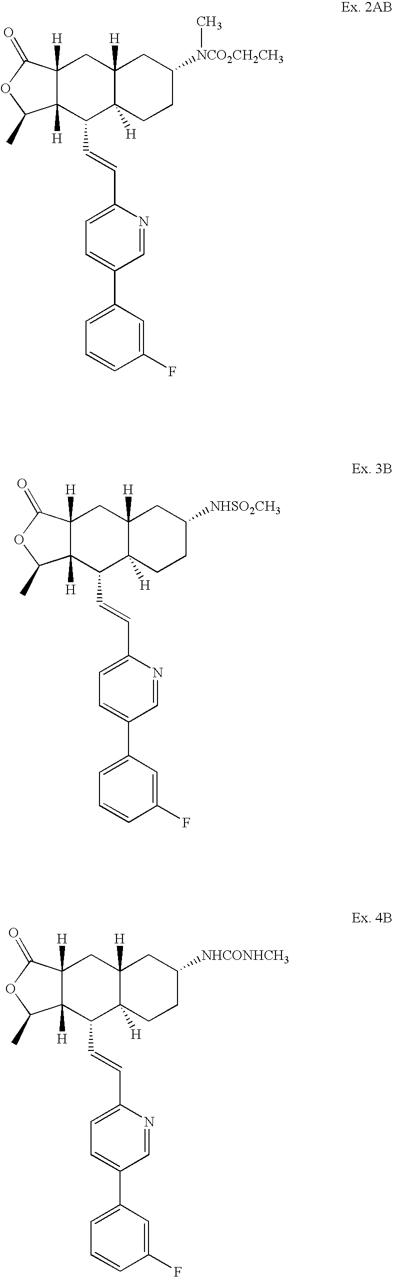

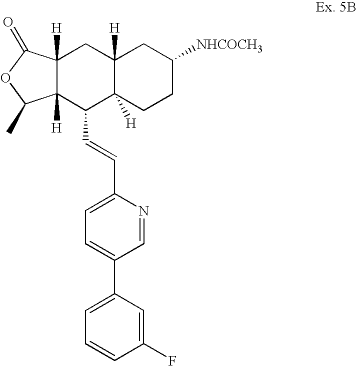

Compounds of the following structure were prepared,

TABLE 4

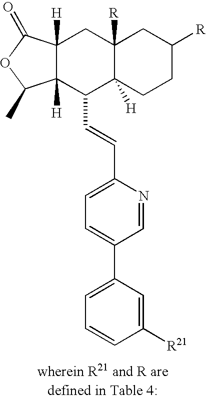

| Ex. | R²¹ | R | Physical data |
|---|---|---|---|
| 6B | —CF₃ | —NHCO₂-t-butyl | MS (M + 1): |
| | | | observed: 571 |
| 7B | —CF₃ | —NHCO₂CH₃ | HRMS (M + 1): |
| | | | observed: 529.2323 |
| 8B | —CF₃ | —NHCO₂CH₂CH₃ | HRMS (M+ 1): |
| | | | observed: 543.2467 |
| 9B | —CF₃ | —NHCO₂CH₂CH₂OCH₃ | HRMS (M + 1): |
| | | | observed: 573.2569 |
| 10B | H | —NHCO₂CH₂CH₃ | HRMS (M + 1): |
| | | | observed: 475.2592 |
| 11B | F | 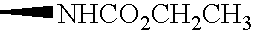 | HRMS (M + 1): observed: 493.2509 |
| 12B* | —CF₃ | —N(n-Pr)CO₂CH₂CH₃ | HRMS (M + 1): |
| | | | observed: 585.2951 |
| 13B* | —CF₃ | —N(n-Pr)CO₂CH₂CH₃ | HRMS (M + 1): |
| | | | observed: 585.2951 |
| 14B | —CF₃ | 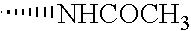 | HRMS (M + 1): observed: 513.2362 |
| 15B | —CF₃ | 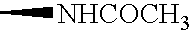 | HRMS (M + 1): observed: 513.2367 |
| 16B | F | 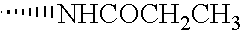 | HRMS (M + 1): observed: 477.2560 |
| 17B | F | 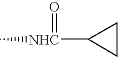 | HRMS (M + 1): observed: 489.2557 |
| 18B | F | 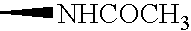 | HRMS (M + 1): observed: 463.2401 |
| 19B | —CF₃ | —NHCOCH₂OCH₃ | HRMS (M + 1): |
| | | | observed: 543.2465 |
| 20B | —CF₃ | —NHCOCH₂OC(O)CH₃ | HRMS (M + 1): |
| | | | observed: 571.2416 |
| 21B | —CF₃ | —NHCONHCH₂CH₃ | HRMS (M + 1): |
| | | | observed: 542.2636 |
| 22B | —CF₃ | —NHCONHCH₃ | HRMS (M + 1): |
| | | | observed: 556.2795 |
| 23B | F | 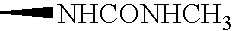 | HRMS (M + 1): observed: 478.2511 |
| 24B | F | —NHCONHCH₂CH₃ | HRMS (M + 1): |
| | | | observed: 492.2669 |
| 25B | F | 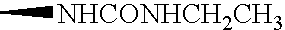 | HRMS (M + 1): observed: 492.2668 |
| 26B | —CF₃ | —NHSO₂CH₃ | HRMS (M + 1): |
| | | | observed: 563.2198 |
| 27B | —CF₃ | —NHSO₂CH₂CH₃ | HRMS (M + 1): |
| | | | observed: 549.2024 |
| 28B | —CF₃ | —NHSO₂CH₂CH₂CH₃ | HRMS (M + 1): |
| | | | observed: 577.2352 |
| 29B | H | —NHSO₂CH₃ | HRMS (M + 1): |
| | | | observed: 481.2164 |
| 30B | —CF₃ | 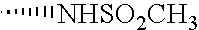 | HRMS (M + 1): observed: 549.2026 |
| 31B | F | 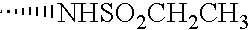 | HRMS (M + 1): observed: 513.2217 |

Replacing the pyridine group of compound 1B with a quinoline group, compounds of the following structure were prepared,

TABLE 5

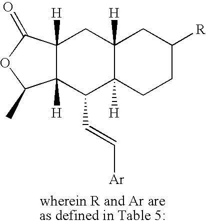

| Ex. | Ar | -R | Physical data |
|---|---|---|---|
| 32B | 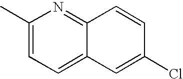 | 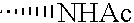 | MS m/z 453 (MH⁺) |
| 33B | 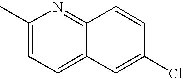 | 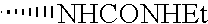 | MS m/z 482 (MH⁺) |
| 34B | 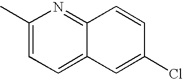 | 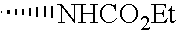 | MS m/z 483 (MH⁺) |
| 35B | 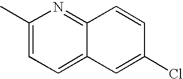 | 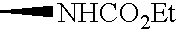 | MS m/z 483 (MH⁺) |
| 36B | 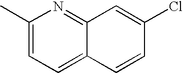 | 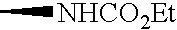 | MS m/z 483 (MH⁺) |
| 37B | 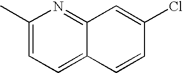 | 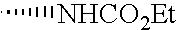 | MS m/z 483 (MH⁺) |
| 38B | 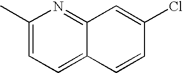 | 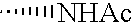 | MS m/z 453 (MH⁺) |
| 40B | 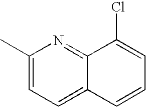 | 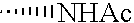 | MS m/z 453 (MH⁺) |
| 41B | 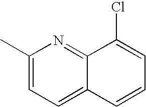 | 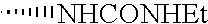 | MS m/z 482 (MH⁺) |
| 42B | 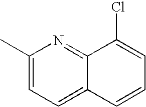 | 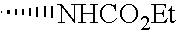 | MS m/z 483 (MH⁺) |
| 43B | 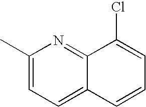 | 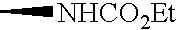 | MS m/z 483 (MH⁺) |

The following analogs were prepared employing further variations of W selected from substituted phenyl and heteroaryl groups:

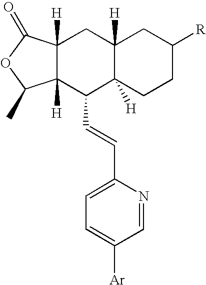

wherein R and Ar are as defined in Table 6:

TABLE 6

| Ex. | Ar | —R | Physical Data |
|---|---|---|---|
| 44B | 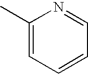 | 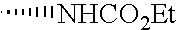 | MS m/z 476 (MH+) |
| 45B | 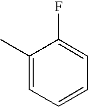 | 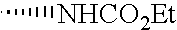 | MS m/z 493 (MH+) |
| 46B | 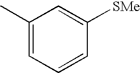 | 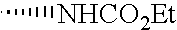 | MS m/z 521 (MH+) |
| 47B | 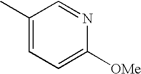 | 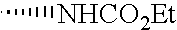 | MS m/z 506 (MH+) |
| 48B | 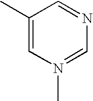 | 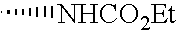 | MS m/z 477 (MH+) |
| 49B | 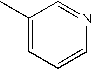 | 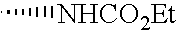 | MS m/z 476 (MH+) |
| 50B | 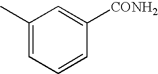 | 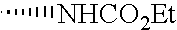 | MS m/z 518 (MH+) |
| 51B | 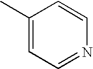 | 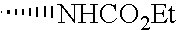 | MS m/z 476 (MH+) |
| 52B | 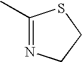 | 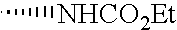 | MS m/z 482 (MH+) |
| 53B | 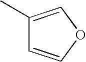 | 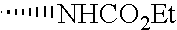 | MS m/z 465 (MH+) |
| 54B | 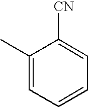 | 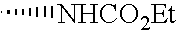 | MS m/z 500 (MH+) |
| 55B | 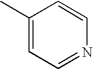 | 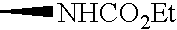 | MS m/z 476 (MH+) |
| 56B | 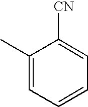 | 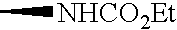 | MS m/z 500 (MH+) |
| 57B | 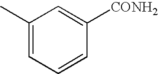 | 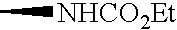 | MS m/z 518 (MH+) |
| 58B | 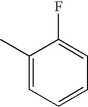 | 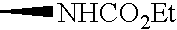 | MS m/z 493 (MH+) |
| 59B | 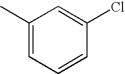 | 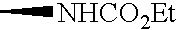 | MS m/z 509 (MH+) |
| 60B | 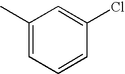 | 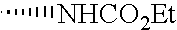 | MS m/z 509 (MH+) |
| 61B | 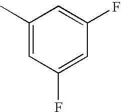 | 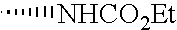 | MS m/z 511 (MH+) |
| 62B | 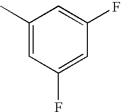 | 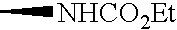 | MS m/z 511 (MH+) |
| 63B | 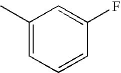 | 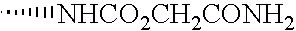 | MS m/z 522 (MH+) |
| 64B | 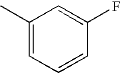 | 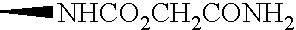 | MS m/z 522 (MH+) |
| 65B | 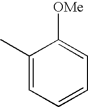 | 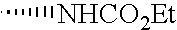 | MS m/z 505 (MH+) |
| 66B | 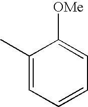 | 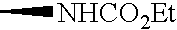 | MS m/z 505 (MH+) |
| 67B | 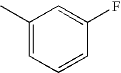 | 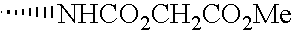 | MS m/z 537 (MH+) |
| 68B | 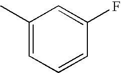 | 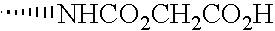 | MS m/z 523 (MH+) |

### EXAMPLE 69B

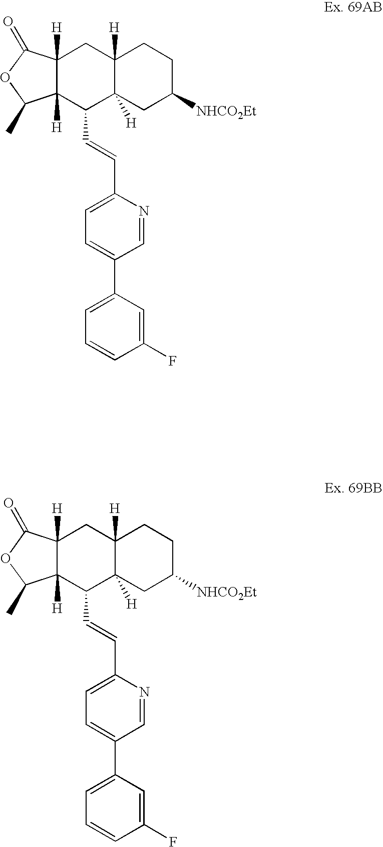

### EXAMPLE 70B

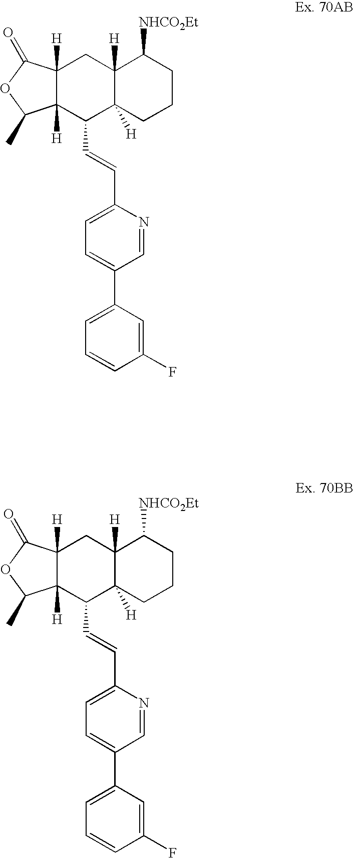

### EXAMPLE 71B

### EXAMPLE 72B

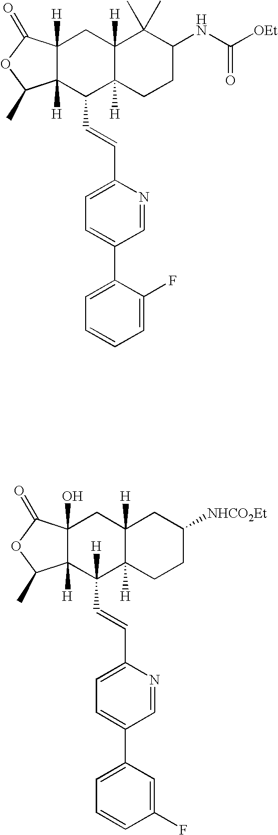

### EXAMPLE 73B

### EXAMPLE 74B

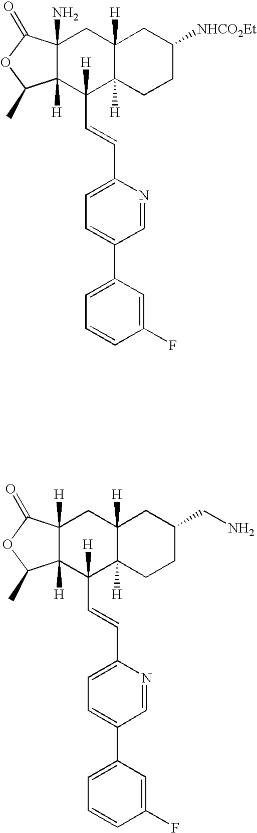

Table 7 discloses compounds of the following structure by displaying definitions of R:

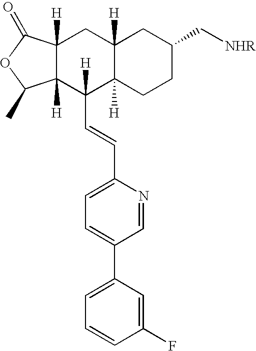

| Ex. | R | HRMS (MH⁺) |
|---|---|---|
| 74B | H | 435.2445 |
| 75B | 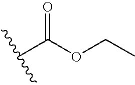 | 507.2664 |
| 76B | 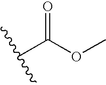 | 493.2497 |
| 77B | 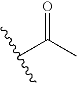 | 477.2548 |
| 78B | 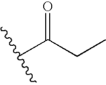 | 491.2703 |
| 79B | 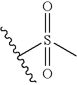 | 513.2213 |
| 80B | 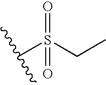 | 527.2388 |
| 81B | 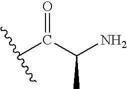 | 506.2822 |
| 82B | 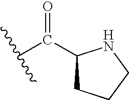 | 532.2970 |
| 83B | 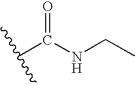 | 506.2822 |
| 84B | 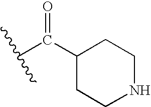 | 546.3124 |

### EXAMPLES 85B-92B

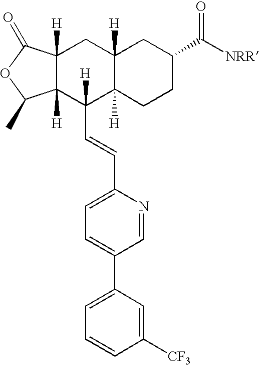

Table 8 discloses compounds of the following structure by displaying definitions of NRR′:

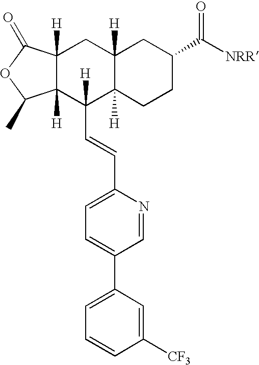

TABLE 8

| EX. | NRR' | (MH+)HRMS |
|---|---|---|
| 85B | 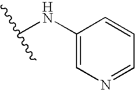 | 576.2481 |
| 86B | 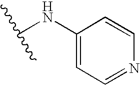 | 576.2472 |
| 87B | 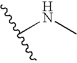 | 513.2370 |
| 88B | 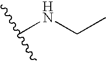 | 527.2517 |
| 89B | 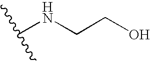 | 543.2477 |
| 90B | 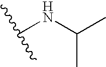 | 541.2669 |
| 91B | 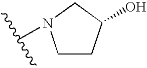 | 569.2632 |
| 92B | 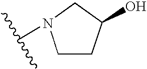 | 569.2627 |

Formulations and Dosing

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), The Science and Practice of Pharmacy, 20^{th} Edition, Lippincott Williams & Wilkins, Baltimore, Md., (2000).

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The daily dose of a compound of Formula I or II for treatment of a disease or condition cited above is about 0.001 to about 100 mg/kg of body weight per day, preferably about 0.001 to about 10 mg/kg. For an average body weight of 70 kg, the dosage level is therefore from about 0.1 to about 700 mg of drug per day, given in a single dose or 2-4 divided doses.

The amount and frequency of administration of the compounds of the invention and/or the pharmaceutically acceptable salts thereof will be regulated according to the judgment of the attending clinician considering such factors as age, condition and size of the patient as well as severity of the symptoms being treated.

Further embodiments of the invention encompass the administration of compounds of Formula I or II along with at least one additional therapeutically effective agent. The contemplated additional therapeutically effective agent is one that differs in either atomic make up or arrangement from the compounds of Formula I or II. Therapeutically effective agents that can be used in combination with the novel compounds of this invention include drugs that are known and used in the treatment of inflammation, rheumatism, asthma, glomerulonephritis, osteoporosis, neuropathy and/or malignant tumors, angiogenesis related disorders, cancer, disorders of the liver, kidney and lung, melanoma, renal cell carcinoma, renal disease, acute renal failure, chronic renal failure, renal vascular homeostasis, glomerulonephritis, chronic airways disease, bladder inflammation, neurodegenerative and/or neurotoxic diseases, conditions, or injuries, radiation fibrosis, endothelial dysfunction, periodontal diseases and wounds. Further examples of therapeutically effective agents which may be administered in combination with the compounds of Formula I or II include resistance factors for tumor cells towards chemotherapy and proliferation inhibitors of smooth muscle cells, endothelial cells, fibroblasts, kidney cells, osteosarcoma cells, muscle cells, cancer cells and/or glial cells. The therapeutically effective agents may be cardiovascular agents.

Cardiovascular agents that can be used in combination with the novel compounds of this invention include drugs that have anti-thrombotic, anti-platelet aggregation, antiatherosclerotic, antirestenotic and/or anti-coagulant activity. Such drugs are useful in treating thrombosis-related diseases including thrombosis, atherosclerosis, restenosis, hypertension, angina pectoris, arrhythmia, heart failure, myocardial infarction, glomerulonephritis, thrombotic and thromboembolic stroke, peripheral vascular diseases, other cardiovascular diseases, cerebral ischemia, inflammatory disorders and cancer, as well as other disorders in which thrombin and its receptor play a pathological role. Suitable cardiovascular agents are selected from the group consisting of thromboxane A2 biosynthesis inhibitors such as aspirin; thromboxane antagonists such as seratrodast, picotamide and ramatroban; adenosine diphosphate (ADP) inhibitors such as clopidogrel; cyclooxygenase inhibitors such as aspirin, meloxicam, rofecoxib and celecoxib; angiotensin antagonists such as valsartan, telmisartan, candesartran, irbesartran, losartan and eprosartan; endothelin antagonists such as tezosentan; phosphodiesterase inhibitors such as milrinoone and enoximone; angiotensin converting enzyme (ACE) inhibitors such as captopril, enalapril, enaliprilat, spirapril, quinapril, perindopril, ramipril, fosinopril, trandolapril, lisinopril, moexipril and benazapril; neutral endopeptidase inhibitors such as candoxatril and ecadotril; anticoagulants such as ximelagatran, fondaparin and enoxaparin; diuretics such as chlorothiazide, hydrochlorothiazide, ethacrynic acid, furosemide and amiloride; platelet aggregation inhibitors such as abciximab and eptifibatide; and GP IIb/IIIa antagonists.

Preferred types of drugs for use in combination with the novel compounds of this invention are thromboxane A2 biosynthesis inhibitors, cyclooxygenase inhibitors and ADP antagonists. Especially preferred for use in the combinations are aspirin and clopidogrel bisulfate.

Further embodiments of the invention encompass the administration of compounds of Formula I or II along with more than one additional therapeutically effective agent. In these embodiments, the additional therapeutically effective agent may or may not be commonly used in the treatment of the same condition. For example, a compound of Formula I or II may be administered along with two cardiovascular agents. Alternatively, a compound of Formula I or II may be administered along with a cardiovascular agent and a therapeutically effective agent useful in the treatment of inflammation.

When the invention comprises a combination of a compound of Formula I or II and one or more other therapeutically effective agents, the two or more active components may be co-administered simultaneously or sequentially, or a single pharmaceutical composition comprising a compound of Formula I or II and the other therapeutically effective agent(s) in a pharmaceutically acceptable carrier can be administered. The components of the combination can be administered individually or together in any conventional dosage form such as capsule, tablet, powder, cachet, suspension, solution, suppository, nasal spray, etc. The dosage of the other therapeutically active agent(s) can be determined from published material, and may range from 1 to 1000 mg per dose.

In this specification, the term “at least one compound of Formula I” means that one to three different compounds of Formula I may be used in a pharmaceutical composition or method of treatment. Preferably one compound of Formula I is used. Similarly, the term “one or more additional cardiovascular agents” means that one to three additional drugs may be administered in combination with a compound of Formula I; preferably, one additional compound is administered in combination with a compound of Formula I. The additional cardiovascular agents can be administered sequentially or simultaneously with reference to the compound of Formula I. The term “at least one compound of Formula II” has a similar meaning with respect to compounds of Formula II.

While the present invention has been described in conjunction with the specific embodiments set forth above, many alternatives, modifications and variations thereof will be apparent to those of ordinary skill in the art. All such alternatives, modifications, and variations are intended to fall within the spirit and scope of the present invention.

We claim:

1. A method of treating a therapeutic condition comprising administering to a mammal in need of such treatment an effective amount of at least one compound of the formula: 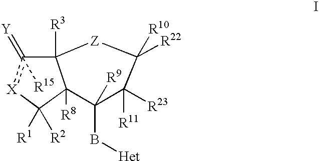
or a pharmaceutically acceptable isomer, salt, solvate or co-crystal form thereof, wherein: 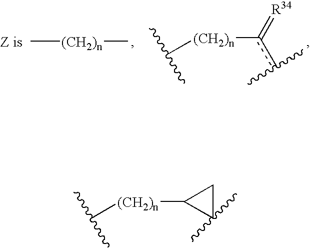
when R¹⁰ is absent, or 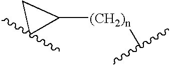
when R is absent;
the single dotted line adjacent to R³⁴ [symbol] represents an optional double bond;
the double dotted lines adjacent to X [symbol] together represent an optional single bond;
n is 0-2;
R¹ and R² are independently selected from the group consisting of H, C₁-C₆ alkyl, fluoro(C₁-C₆)alkyl, difluoro(C₁-C₆)alkyl, trifluoro-(C₁-C₆)alkyl, C₃-C₇ cycloalkyl, C₂-C₆ alkenyl, aryl(C₁-C₆)alkyl, aryl(C₂-C₆)alkenyl, heteroaryl(C₁-C₆)alkyl, heteroaryl(C₂-C₆)alkenyl, hydroxy-(C₁-C₆)alkyl, (C₁-C₆)alkoxy(C₁-C₆)alkyl, amino-(C₁-C₆)alkyl, aryl and thio(C₁-C₆)alkyl; or R¹ and R² together form a ═O group;
R³ is H, hydroxy, C₁-C₆ alkoxy, —NR¹⁸R¹⁹, —SOR¹⁶, —SO₂R¹⁷, —C(O)OR¹⁷, —C(O)NR¹⁸R¹⁹, C₁-C₆ alkyl, halogen, fluoro(C₁-C₆)alkyl, difluoro(C₁-C₆)alkyl, trifluoro(C₁-C₆)alkyl, C₃-C₇ cycloalkyl, C₂-C₆ alkenyl, aryl(C₁-C₆)alkyl, aryl(C₂-C₆)alkenyl, heteroaryl(C₁-C₆)alkyl, heteroaryl(C₂-C₆)alkenyl, hydroxy(C₁-C₆)alkyl, amino(C₁-C₆)alkyl, aryl, thio(C₁-C₆)alkyl, (C₁-C₆)alkoxy(C₁-C₆)alkyl or (C₁-C₆)alkylamino(C₁-C₆)alkyl;
R³⁴ is (H, R³), (H, R⁴³), ═O or ═NOR¹⁷ when the optional double bond adjacent to R³⁴ is absent; R³⁴ is R⁴⁴ when the double bond is present;
Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member of Het, and wherein the Het group is substituted by 1 to 4 moieties, W, independently selected from the group consisting of H; C₁-C₆ alkyl; fluoro(C₁-C₆)alkyl; difluoro(C₁-C₆)alkyl; trifluoro-(C₁-C₆)-alkyl; C₃-C₇ cycloalkyl; heterocycloalkyl; heterocycloalkyl substituted by C₁-C₆ alkyl, C₂-C₆ alkenyl, OH—(C₁-C₆)alkyl, or ═O; C₂-C₆ alkenyl; R²¹-aryl(C₁-C₆)alkyl; R²¹-aryl-(C₂-C₆)-alkenyl; R²¹-aryloxy; R²¹-aryl-NH—; heteroaryl(C₁-C₆)alkyl; heteroaryl(C₂-C₆)-alkenyl; heteroaryloxy; heteroaryl-NH—; hydroxy(C₁-C₆)alkyl; dihydroxy(C₁-C₆)alkyl; amino(C₁-C₆)alkyl; (C₁-C₆)alkylamino-(C₁-C₆)alkyl; di-((C₁-C₆)alkyl)-amino(C₁-C₆)alkyl; thio(C₁-C₆)alkyl; C₁-C₆ alkoxy; C₂-C₆ alkenyloxy; halogen; —NR⁴R⁵; —CN; —OH; —COOR¹⁷; —COR¹⁶; —OSO₂CF₃; —CH₂OCH₂CF₃; (C₁-C₆)alkylthio; —C(O)NR⁴R⁵; —OCHR⁶-phenyl; phenoxy-(C₁-C₆)alkyl; —NHCOR¹⁶; —NHSO₂R¹⁶; biphenyl; —OC(R⁶)₂COOR⁷; —OC(R⁶)₂C(O)NR⁴R⁵; (C₁-C₆)alkoxy; —C(═NOR¹⁷)R¹⁸; C₁-C₆ alkoxy substituted by (C₁-C₆)alkyl, amino, —OH, COOR¹⁷, —NHCOOR¹⁷, —CONR⁴R⁵, aryl, aryl substituted by 1 to 3 moieties independently selected from the group consisting of halogen, —CF₃, C₁-C₆ alkyl, C₁-C₆ alkoxy and —COOR¹⁷, aryl wherein adjacent carbons form a ring with a methylenedioxy group, —C(O)NR⁴R⁵ or heteroaryl; R²¹-aryl; aryl wherein adjacent carbons form a ring with a methylenedioxy group; R⁴¹-heteroaryl; and heteroaryl wherein adjacent carbon atoms form a ring with a C₃-C₅ alkylene group or a methylenedioxy group;
R⁴ and R⁵ are independently selected from the group consisting of H, C₁-C₆ alkyl, phenyl, benzyl and C₃-C₇ cycloalkyl, or R⁴ and R⁵ together are —(CH₂)₄—, —(CH₂)5— or —(CH₂)₂NR⁷—(CH₂)₂— and form a ring with the nitrogen to which they are attached;
R⁶ is independently selected from the group consisting of H, C₁-C₆ alkyl, phenyl, (C₃-C₇)cycloalkyl, (C₃-C₇)cycloalkyl(C₁-C₆)alkyl, (C₁-C₆)alkoxy(C₁-C₆)alkyl, hydroxy(C₁-C₆)alkyl and amino(C₁-C₆)alkyl;
R⁷ is H or (C₁-C₆)alkyl;
R⁸, R¹⁰ and R¹¹ are independently selected from the group consisting of R¹ and —OR¹, provided that when the optional double bond is present, R¹⁰ is absent;
R⁹ is H, OH, C₁-C₆ alkoxy, halogen or halo(C₁-C₆)alkyl;
B is —(CH₂)ₙ₃—, —CH₂—O—, —CH₂S—, —CH₂—NR⁶—, —C(O)NR⁶—, —NR⁶C(O)—, [symbol] cis or trans —(CH₂)ₙ₄CR¹²═CR^{12a}(CH₂)ₙ₅— or —(CH₂)ₙ₄C≡C(CH₂)ₙ₅—, wherein n₃ is 0-5, n₄ and n₅ are independently 0-2, and R¹² and R^{12a} are independently selected from the group consisting of H, C₁-C₆ alkyl and halogen;
X is —O— or —NR⁶— when the double dotted lines adjacent to X represent a single bond, or X is H, —OH or —NHR²⁰ when the bond is absent;
Y is ═O, ═S, (H, H), (H, OH) or (H, C₁-C₆ alkoxy) when the double dotted lines adjacent to X represent a single bond, or when the bond is absent, Y is ═O, ═NOR¹⁷, (H, H), (H, OH), (H, SH), (H, C₁-C₆ alkoxy) or (H, —NHR⁴⁵);
R¹⁵ is absent when the double dotted lines adjacent to X represent a single bond; R¹⁵ is H, C₁-C₆ alkyl, —NR¹⁸R¹⁹ or —OR¹⁷ when said single bond is absent; or Y is 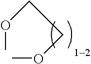
or 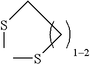
and R¹⁵ is H or C₁-C₆ alkyl;
R¹⁶ is C₁-C₆ lower alkyl, phenyl or benzyl;
R¹⁷, R¹⁸ and R¹⁹ are independently selected from the group consisting of H, C₁-C₆ alkyl, phenyl, benzyl;
R²⁰ is H, C₁-C₆ alkyl, phenyl, benzyl, —C(O)R⁶ or —SO₂R⁶;
R²¹ is 1 to 3 moieties independently selected from the group consisting of hydrogen, —CN, —CF₃, —OCF₃, halogen, —NO₂, C₁-C₆ alkyl, C₁-C₆alkoxy, (C₁-C₆)alkylamino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —NHCOR¹⁶, —NHSO₂R¹⁶, —NHSO₂CH₂CF₃, heteroaryl or —C(═NOR¹⁷)R¹⁸;
R²² and R²³ are independently selected from the group consisting of hydrogen, R²⁴—(C₁-C₁₀)alkyl, R²⁴⁻(C₂-C₁₀)alkenyl, R²⁴—(C₂-C₁₀)alkynyl, R²⁷-hetero-cycloalkyl, R²⁵-aryl, R²⁵-aryl(C₁-C₆)alkyl, R²⁹—(C₃-C₇)cycloalkyl, R²⁹—(C₃-C₇)cycloalkenyl, —OH, —OC(O)R³⁰, —C(O)OR³⁰, —C(O)R³⁰, —C(O)NR³⁰OR³¹, —NR³⁰R³¹, —NR³⁰C(O)R³¹, —NR³⁰C(O)NR³¹R³², —NHSO₂R³⁰, —OC(O)NR³⁰R³¹, R²⁴—(C₁-C₁₀)alkoxy, R²⁴—(C₂-C₁₀)-alkenyloxy, R²⁴—(C₂-C₁₀)alkynyloxy, R²⁷-heterocycloalkyloxy, R²⁹—(C₃-C₇)cycloalkyloxy, R²⁹—(C₃-C₇)cyclo-alkenyloxy, R²⁹—(C₃-C₇)cycloalkyl-NH—, —CH₂—O—CH₂-phenyl, —NHSO₂NHR¹⁶ and —CH(═NOR¹⁷);
or R²² and R¹⁰ together with the carbon to which they are attached, or R²³ and R¹¹ together with the carbon to which they are attached, independently form a R⁴²-substituted carbocyclic ring of 3-10 atoms, or a R⁴²-substituted heterocyclic ring of 4-10 atoms wherein 1-3 ring members are independently selected from the group consisting of —O—, —NH— and —SO₀₋₂—, provided that when R²² and R¹⁰ form a ring, the optional double bond is absent;
R²⁴ is 1, 2 or 3 moieties independently selected from the group consisting of hydrogen, halogen, —OH, (C₁-C₆)alkoxy, R³⁵-aryl, (C₁-C₁₀)-alkyl-C(O)—, (C₂-C₁₀)-alkenyl-C(O)—, (C₂-C₁₀)alkynyl-C(O)—, heterocycloalkyl, R²⁶—(C₃-C₇)cycloalkyl, R²⁶—(C₃-C₇)cycloalkenyl, —OC(O)R³⁰, —C(O)OR³⁰, —C(O)R³⁰, —C(O)NR³⁰OR³¹, —NR³⁰R³¹, —NR³⁰C(O)R³¹, —NR³⁰C(O)NR³¹R³², —NHSO₂R³⁰, —OC(O)NR³⁰R³¹, R²⁴—(C₂-C₁₀)-alkenyloxy, R²⁴—(C₂-C₁₀)alkynyloxy, R²⁷-heterocycloalkyloxy, R²⁹—(C₃-C₇)-cycloalkyloxy, R²⁹—(C₃-C₇)cyclo-alkenyloxy, R²⁹—(C₃-C₇)cycloalkyl-NH—, —NHSO₂NHR¹⁶ and —CH(═NOR¹⁷);
R²⁵ is 1, 2 or 3 moieties independently selected from the group consisting of hydrogen, heterocycloalkyl, halogen, —COOR³⁶, —CN, —C(O)NR³⁷R³⁸, —NR³⁹C(O)R⁴⁰, —OR³⁶, (C₃-C₇)cycloalkyl, (C₃-C₇)cycloalkyl-C₁-C₆)alkyl, (C₁-C₆)alkyl(C₃-C₇)cycloalkyl-(C₁-C₆)alkyl, halo(C₁-C₆)alkyl(C₃-C₇)cycloalkyl(C₁-C₆)alkyl, hydroxy(C₁-C₆)alkyl, (C₁-C₆)alkoxy(C₁-C₆)alkyl, and R⁴¹-heteroaryl; or two R²⁵ groups on adjacent ring carbons form a fused methylenedioxy group;
R²⁶ is 1, 2, or 3 moieties independently selected from the group consisting of hydrogen, halogen and (C₁-C₆)alkoxy;
R²⁷ is 1, 2 or 3 moieties independently selected from the group consisting of hydrogen, R²⁸—(C₁-C₁₀)alkyl, R²⁸—(C₂-C₁₀)alkenyl, R²⁸—(C₂-C₁₀)alkynyl;
R²⁸ is hydrogen, —OH or (C₁-C₆)alkoxy;
R²⁹ is 1, 2 or 3 moieties independently selected from the group consisting of hydrogen, (C₁-C₆)alkyl, —OH, (C₁-C₆)alkoxy and halogen;
R³⁰, R³¹ and R³² are independently selected from the group consisting of hydrogen, (C₁-C₁₀)-alkyl, (C₁-C₆)alkoxy(C₁-C₁₀)-alkyl, R²⁵-aryl(C₁-C₆)-alkyl, R³³—(C₃-C₇)cycloalkyl, R³⁴⁻(C₃-C₇)cycloalkyl(C₁-C₆)alkyl, R²⁵-aryl, heterocycloalkyl, heteroaryl, heterocycloalkyl(C₁-C₆)alkyl and heteroaryl(C₁-C₆)alkyl;
R³³ is hydrogen, (C₁-C₆)alkyl, OH—(C₁-C₆)alkyl or (C₁-C₆)alkoxy;
R³⁵ is 1 to 4 moieties independently selected from the group consisting of hydrogen, (C₁-C₆)alkyl, —OH, halogen, —CN, (C₁-C₆)alkoxy, trihalo(C₁-C₆)alkoxy, (C₁-C₆)alkylamino, di((C₁-C₆)alkyl)amino, —OCF₃, OH—(C₁-C₆)alkyl, —CHO, —C(O)(C₁-C₆)-alkylamino, —C(O)di((C₁-C₆)alkyl)amino, —NH₂, —NHC(O)(C₁-C₆)alkyl and —N((C₁-C₆)alkyl)C(O)(C₁-C₆)alkyl;
R³⁶ is hydrogen, (C₁-C₆)alkyl, halo(C₁-C₆)alkyl, dihalo(C₁-C₆)alkyl or trifluoro(C₁-C₆)alkyl;
R³⁷ and R³⁸ are independently selected from the group consisting of hydrogen, (C₁-C₆)alkyl, aryl(C₁-C₆)alkyl, phenyl and (C₃-C₁₅)cycloalkyl, or R³⁷ and R³⁸ together are —(CH₂)₄—, —(CH₂)₅— or —(CH₂)₂—NR³⁹—(CH₂)₂— and form a ring with the nitrogen to which they are attached;
R³⁹ and R⁴⁰ are independently selected from the group consisting of hydrogen, (C₁-C₆)alkyl, aryl(C₁-C₆)alkyl, phenyl and (C₃-C₁₅)-cycloalkyl, or R³⁹ and R⁴⁰ in the group —NR³⁹C(O)R⁴⁰, together with the carbon and nitrogen atoms to which they are attached, form a cyclic lactam having 5-8 ring members;
R⁴¹ is 1 to 4 moieties independently selected from the group consisting of hydrogen, (C₁-C₆)alkyl, (C₁-C₆)alkoxy, (C₁-C₆)alkylamino, di((C₁-C₆)alkyl)amino, —OCF₃, OH—(C₁-C₆)alkyl, —CHO and phenyl;
R⁴² is 1 to 3 moieties independently selected from the group consisting of hydrogen, —OH, (C₁-C₆)alkyl and (C₁-C₆)alkoxy;
R⁴³ is —NR³⁰R³¹, —NR³⁰C(O)R³¹, —NR³⁰C(O)NR³¹R³², —NHSO₂R³⁰ or —NHCOOR¹⁷;
R⁴⁴ is H, C₁-C₆ alkoxy, —SOR¹⁶, —SO₂R¹⁷, —C(O)OR¹⁷, —C(O)NR¹⁸R¹⁹, C₁-C₆ alkyl, halogen, fluoro(C₁-C₆)alkyl, difluoro(C₁-C₆)alkyl, trifluoro(C₁-C₆)alkyl, C₃-C₇ cycloalkyl, C₂-C₆ alkenyl, aryl(C₁-C₆)alkyl, aryl(C₂-C₆)alkenyl, heteroaryl(C₁-C₆)alkyl, heteroaryl(C₂-C₆)alkenyl, hydroxy(C₁-C₆)alkyl, amino(C₁-C₆)alkyl, aryl, thio(C₁-C₆)alkyl, (C₁-C₆)alkoxy(C₁-C₆)alkyl or (C₁-C₆)alkylamino(C₁-C₆)alkyl; and
R⁴⁵ is H, C₁-C₆ alkyl, —COOR¹⁶ or —SO₂,
wherein said therapeutic condition is a cardiovascular or circulatory disease or condition, an inflammatory disease or condition, a respiratory tract disease or condition, cancer, acute renal failure, glomerulonephritis, astrogliosis, a fibrotic disorder of the liver, kidney, lung or intestinal tract, Alzheimer's disease, diabetes, diabetic neuropathy, rheumatoid arthritis, neurodegenerative disease, neurotoxic disease, systemic lupus erythematosus, multiple sclerosis, osteoporosis, glaucoma, macular degeneration, psoriasis, radiation fibrosis, endothelial dysfunction, a wound or a spinal cord injury, or a symptom or result thereof.

2. The method of claim 1 wherein the cardiovascular or circulatory disease or condition is atherosclerosis, restenosis, hypertension, acute coronary syndrome, angina pectoris, arrhythmia, heart disease, heart failure, myocardial infarction, thrombotic or thromboembolytic stroke, a peripheral vascular disease, deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, renal vascular homeostasis or erectile dysfunction.

3. The method of claim 1 wherein the inflammatory disease or condition is irritable bowel syndrome, Crohn's disease, nephritis or a radiation- or chemotherapy-induced proliferative or inflammatory disorder of the gastrointestinal tract, lung, urinary bladder, gastrointestinal tract or other organ.

4. The method of claim 1 wherein the respiratory tract disease or condition is reversible airway obstruction, asthma, chronic asthma, bronchitis or chronic airways disease.

5. The method of claim 1 wherein the cancer is renal cell carcinoma or an angiogenesis related disorder.

6. The method of claim 1 wherein the neurodegenerative disease is Parkinson's disease, amyotropic lateral sclerosis, Alzheimer's disease, Huntington's disease or Wilson's disease.

7. The method of claim 1 further comprising administering at least one therapeutically effective agent useful in the treatment of inflammation, rheumatism, asthma, glomerulonephritis, osteoporosis, neuropathy and/or malignant tumors, angiogenesis related disorders, cancer, disorders of the liver, kidney or lung, melanoma, renal cell carcinoma, renal disease, acute renal failure, chronic renal failure, renal vascular homeostasis, glomerulonephritis, chronic airways disease, bladder inflammation, neurodegenerative and/or neurotoxic diseases, conditions, or injuries, radiation fibrosis, endothelial dysfunction, periodontal diseases or wounds.

8. The method of claim 7 further comprising administering at least two therapeutically effective agents.

9. A method of treating a therapeutic condition comprising administering to a mammal in need of such treatment an effective amount of at least one compound of the formula: 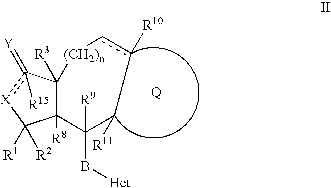
or a pharmaceutically acceptable isomer, salt, solvate or co-crystal form thereof, wherein:
the double dotted lines adjacent to X [symbol] together represent an optional single bond;
the single dotted line adjacent to R¹⁰ [symbol] represents an optional double bond;
n is 0-2;
Q is 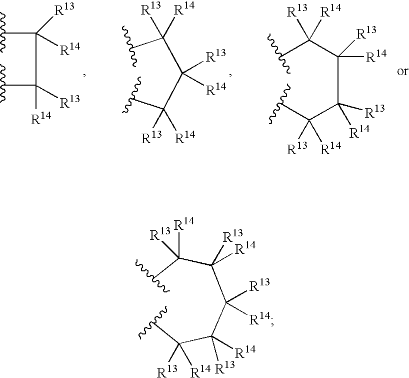
R¹ and R² are independently selected from the group consisting of H, (C₁-C₆)alkyl, fluoro(C₁-C₆)alkyl-, difluoro(C₁-C₆)alkyl-, trifluoro-(C₁-C₆)alkyl-, (C₃-C₆)cycloalkyl, (C₂-C₆)alkenyl, hydroxy-(C₁-C₆)alkyl-, and amino(C₁-C₆)alkyl-;
R³ is H, hydroxy, (C₁-C₆)alkoxy, —SOR¹⁶, —SO₂R¹⁷, —C(O)OR¹⁷, —C(O)NR¹⁸R¹⁹, —(C₁-C₆)alkyl-C(O)NR¹⁸R¹⁹, (C₁-C₆)alkyl, halogen, fluoro(C₁-C₆)alkyl-, difluoro(C₁-C₆)alkyl-, trifluoro(C₁-C₆)alkyl-, (C₃-C₆)cycloalkyl, (C₃-C₆)-cycloalkyl-(C₁-C₆)alkyl-, (C₂-C₆)alkenyl, aryl(C₁-C₆)alkyl-, aryl(C₂-C₆)alkenyl-, heteroaryl(C₁-C₆)alkyl-, heteroaryl(C₂-C₆)alkenyl-, hydroxy(C₁-C₆)-alkyl-, —NR²²R²³, NR²²R²³—(C₁-C₆)alkyl-, aryl, thio(C₁-C₆)alkyl-, (C₁-C₆)alkyl-thio(C₁-C₆)alkyl-, (C₁-C₆)alkoxy(C₁-C₆)alkyl-, NR¹⁸R¹⁹—C(O)—(C₁-C₆)alkyl- or (C₃-C₆)cycloalkyl-(C₁-C₆)alkyl-;
Het is a mono- or bi-cyclic heteroaryl group of 5 to 10 atoms comprised of 1 to 9 carbon atoms and 1 to 4 heteroatoms independently selected from the group consisting of N, O and S, wherein a ring nitrogen can form an N-oxide or a quaternary group with a (C₁-C₄)alkyl group, wherein Het is attached to B by a carbon atom ring member of said Het, and wherein the Het group is substituted by W;
W is 1 to 4 moieties independently selected from the group consisting of H, (C₁-C₆)alkyl, fluoro(C₁-C₆)alkyl-, difluoro(C₁-C₆)alkyl-, trifluoro(C₁-C₆)alkyl-, (C₃-C₆)cycloalkyl, hydroxy(C₁-C₆)alkyl-, dihydroxy(C₁-C₆)alkyl-, NR²⁵R²⁶(C₁-C₆)alkyl-, thio(C₁-C₆)alkyl-, —OH, (C₁-C₆)alkoxy, halogen, —NR⁴R⁵, —C(O)OR¹⁷, —COR¹⁶, (C₁-C₆)alkylthio-, R²¹-aryl, R²¹-aryl(C₁-C₆)alkyl-, aryl wherein adjacent ring carbons in said aryl, along with two O atoms, form a methylenedioxy group, and R²¹-heteroaryl;
R⁴ and R⁵ are independently selected from the group consisting of H, (C₁-C₆)alkyl, phenyl, benzyl and (C₃-C₆)cycloalkyl, or R⁴ and R⁵ taken together are —(CH₂)₄—, —(CH₂)₅— or —(CH₂)₂NR⁷—(CH₂)₂— and form a ring with the nitrogen to which they are attached;
R⁶ is H, (C₁-C₆)alkyl or phenyl;
R⁷ is H, (C₁-C₆)alkyl, —C(O)—R¹⁶, —C(O)OR¹⁷ or —S(O)₂R¹⁷;
R⁸, R¹⁰ and R¹¹ are independently selected from the group consisting of R¹ and —OR¹, provided that when the optional double bond shown in Formula II is present, R¹⁰ is absent;
R⁹ is H, OH or (C₁-C₆)alkoxy;
B is —(CH₂)ₙ₃—, cis or trans —(CH₂)ₙ₄CR¹²═CR^{12a}(CH₂)ₙ₅— or —(CH₂)ₙ₄C═C(CH₂)ₙ₅—, wherein n₃ is 0-5, n₄ and n₅ are independently 0-2, and R¹² and R^{12a} are independently selected from the group consisting of H, (C₁-C₆)alkyl and halogen;
X is —O— or —NR⁶— when the dotted line shown adjacent to X in Formula II represents a single bond, or X is —OH or —NHR²⁰ when the bond is absent;
Y is ═O, ═S, (H, H), (H, OH) or (H, (C₁-C₆)alkoxy) when the dotted line shown adjacent to X in Formula II represents a single bond, or when the bond is absent, Y is ═O, (H, H), (H, OH), (H, SH) or (H, (C₁-C₆)alkoxy);
each R¹³ is independently selected from H, (C₁-C₆)alkyl, (C₃-C₈)cycloalkyl, —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4, haloalkyl, and halogen;
each R¹⁴ is independently selected from H, (C₁-C₆)alkyl, —OH, (C₁-C₆)alkoxy, R²⁷-aryl(C₁-C₆)alkyl, heteroaryl, heteroarylalkyl, heterocyclyl, heterocyclylalkyl, —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4, halogen and haloalkyl; or
R¹³ and R¹⁴ taken together form a spirocyclic or a heterospirocyclic ring of 3-6 atoms;
wherein at least one of R¹³ or R¹⁴ is selected from the group consisting of —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4;
R¹⁵ is H when the double dotted line shown adjacent to X in Formula II represents a single bond and is H, (C₁-C₆)alkyl, —NR¹⁸R¹⁹, or —OR¹⁷ when said bond is absent;
R¹⁶ is independently selected from the group consisting of (C₁-C₆)alkyl, phenyl and benzyl;
R^{16b} is H, alkoxy, (C₁-C₆)alkyl, (C₁-C₆)alkoxy(C₁-C₆)alkyl-, R₂₂—O—C(O)—(C₁-C₆)alkyl-, (C₃-C₆)cycloalkyl, R²¹-aryl, R²¹-aryl(C₁-C₆)alkyl, haloalkyl, alkenyl, halosubstituted alkenyl, alkynyl, halosubstituted alkynyl, R²¹-heteroaryl, R²¹—(C₁-C₆)alkyl heteroaryl, R²¹—(C₁-C₆)alkyl heterocycloalkyl, R²⁸R²⁹N—(C₁-C₆)alkyl, R²⁸R²⁹N—(CO)—(C₁-C₆)alkyl, R²⁸R²⁹N—(CO)O—(C₁-C₆)alkyl, R²⁸O(CO)N(R²⁹)—(C₁-C₆)alkyl, R²⁸S(O)₂N(R²⁹)—(C₁-C₆)alkyl, R²⁸R²⁹N—(CO)—N(R²⁹)—(C₁-C₆)alkyl, R²⁸R²⁹N—S(O)2N(R²⁹)—(C₁-C₆)alkyl, R²⁸—(CO)N(R²⁹)—(C₁-C₆)alkyl, R²⁸R²⁹N—S(O)₂—(C₁-C₆)alkyl, HOS(O)₂—(C₁-C₆)alkyl, (OH)₂P(O)₂—(C₁-C₆)alkyl, R₂₈—S—(C₁-C₆)alkyl, R²⁸—S(O)₂—(C₁-C₆)alkyl or hydroxy(C₁-C₆)alkyl);
R¹⁷, R¹⁸ and R¹⁹ are independently selected from the group consisting of H, (C₁-C₆)alkyl, phenyl, and benzyl;
R²⁰ is H, (C₁-C₆)alkyl, phenyl, benzyl, —C(O)R⁶ or —S(O)₂R⁶;
R²¹ is 1 to 3 moieties independently selected from the group consisting of H, —CN, —CF₃, —OCF₃, halogen, —NO₂, (C₁-C₆)alkyl, —OH, (C₁-C₆)alkoxy, (C₁-C₆)-alkylamino-, di-((C₁-C₆)alkyl)amino-, NR²⁵R²⁶⁻(C₁-C₆)alkyl-, hydroxy-(C₁-C₆)alkyl-,—C(O)OR¹⁷, —C(O)R¹⁷, —NHC(O)R¹⁶, —NHS(O)₂R¹⁶, —NHS(O)₂CH₂CF₃, —C(O)NR²⁵R²⁶, —NR²⁵—C(O)—NR²⁵R²⁶, —S(O)R¹³, —S(O)₂R¹³ and —SR¹³;
R²² is H or (C₁-C₆)alkyl;
R²³ is H, (C₁-C₆)alkyl, —C(O)R²⁴, —S(O)₂R²⁴, —C(O)NHR²⁴ or —S(O)₂NHR²⁴;
R²⁴ is (C₁-C₆)alkyl, hydroxy (C₁-C₆)alkyl or NR²⁵R²⁶—((C₁-C₆)alkyl)-;
R²⁵ and R²⁵ are independently selected from the group consisting of H and (C₁-C₆)alkyl;
R²⁷ is 1, 2 or 3 moieties selected from the group consisting of H, (C₁-C⁶)alkyl, (C₃-C₆)cycloalkyl, (C₁-C₆)alkoxy, halogen and —OH; and
R²⁸ and R²⁹ are independently selected from the group consisting of H, (C₁-C₆)alkyl, (C₁-C₆)alkoxy, R²⁷-aryl(C₁-C₆)alkyl, heteroaryl, heteroarylalkyl, hydroxy(C₁-C₆)alkyl, (C₁-C₆)alkoxy(C₁-C₆)alkyl, heterocyclyl, heterocyclylalkyl, and haloalkyl; or
R²⁸ and R²⁹ taken together form a spirocyclic or a heterospirocyclic ring of 3-6 atoms,
wherein said therapeutic condition is a cardiovascular or circulatory disease or condition, an inflammatory disease or condition, a respiratory tract disease or condition, cancer, acute renal failure, glomerulonephritis, astrogliosis, a fibrotic disorder of the liver, kidney, lung or intestinal tract, Alzheimer's disease, diabetes, diabetic neuropathy, rheumatoid arthritis, neurodegenerative disease, neurotoxic disease, systemic lupus erythematosus, multiple sclerosis, osteoporosis, glaucoma, macular degeneration, psoriasis, radiation fibrosis, endothelial dysfunction, a wound or a spinal cord injury, or a symptom or result thereof.

10. The method of claim 9 wherein the cardiovascular or circulatory disease or condition is atherosclerosis, restenosis, hypertension, acute coronary syndrome, angina pectoris, arrhythmia, heart disease, heart failure, myocardial infarction, thrombotic or thromboembolytic stroke, a peripheral vascular disease, deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, renal vascular homeostasis or erectile dysfunction.

11. The method of claim 9 wherein the inflammatory disease or condition is irritable bowel syndrome, Crohn's disease, nephritis or a radiation- or chemotherapy-induced proliferative or inflammatory disorder of the gastrointestinal tract, lung, urinary bladder, gastrointestinal tract or other organ.

12. The method of claim 9 wherein the respiratory tract disease or condition is reversible airway obstruction, asthma, chronic asthma, bronchitis or chronic airways disease.

13. The method of claim 9 wherein the cancer is renal cell carcinoma or an angiogenesis related disorder.

14. The method of claim 9 wherein the neurodegenerative disease is Parkinson's disease, amyotropic lateral sclerosis, Alzheimer's disease, Huntington's disease or Wilson's disease.

15. The method of claim 9 further comprising administering at least one therapeutically effective agent useful in the treatment of inflammation, rheumatism, asthma, glomerulonephritis, osteoporosis, neuropathy and/or malignant tumors, angiogenesis related disorders, cancer, disorders of the liver, kidney or lung, melanoma, renal cell carcinoma, renal disease, acute renal failure, chronic renal failure, renal vascular homeostasis, glomerulonephritis, chronic airways disease, bladder inflammation, neurodegenerative and/or neurotoxic diseases, conditions, or injuries, radiation fibrosis, endothelial dysfunction, periodontal diseases or wounds.

16. The method of claim 15 further comprising administering at least two therapeutically effective agents.
